(19) United States
(12) Patent Application Publication
(10) Pub. No.: US 2024/0167038 A1
(43) Pub. Date: May 23, 2024

(54) RNA Trans-Splicing Molecule

(71) Applicant: Cambridge Enterprise Limited, Cambridge, Cambridgeshire (GB)

(72) Inventors: Carin Kristina Ingemarsdotter, Cambridge (GB); Andrew Michael Lindsay Lever, Cambridge (GB)

(21) Appl. No.: 18/276,848

(22) PCT Filed: Feb. 11, 2022
(86) PCT No.: PCT/EP2022/053407

§ 371 Date: Aug. 10, 2023

(30) Foreign Application Priority Data
Feb. 15, 2021 (GB) 2102118.3

(51) Int. Cl. C12N 15/113; C12N 9/12; C12N 15/52

(52) U.S. Cl. CPC C12N 15/1131; C12N 9/1211; C12N 15/52; C12Y 207/01021; C12N 2310/3519; C12N 2320/33; C12N 2840/445

(57) ABSTRACT

This invention relates to an RNA trans-splicing molecule (RTM) that targets human endogenous retrovirus (HERV) pre-mRNA. The RTM comprises (i) a binding region specific for a HERV pre-mRNA, (ii) a trans-splicing splice domain and (ill) a coding sequence for a suicide protein. The binding region of the RTM binds to HERV pre-mRNA in a cell, such that the coding sequence is trans-spliced through the trans- splicing domain with the HERV pre-mRNA, resulting in a chimeric mRNA causing the suicide protein to be expressed in the cell. RTMs of the invention may be useful in selectively killing cells that express HERV genes, for example cancer cells. RTMs, encoding nucleic acids, methods of treatment and associated methods and uses are provided.

### FIELD

The present invention relates to RNA trans-splicing molecules (RTMs), in particular RTMs which mediate trans-splicing of a suicide gene, and their use in the treatment of cancer.

### BACKGROUND

RNA trans-splicing is a spliceosome-mediated process in which two different RNA molecules are spliced together to generate a chimeric mRNA molecule in the nucleus. After nuclear export, the chimeric mRNA molecule is translated in the cytoplasm to produce a chimeric protein.

RNA trans-splicing has been used to exchange a defective RNA transcript with a corrected mRNA molecule delivered in trans (Hong et al (2020) Br Med Bull. 2020 Dec. 15; 136(1):4-20). RNA trans-splicing has also been used to deliver the two-step Herpes simplex virus thymidine kinase-ganciclovir (HSV-tk/GCV) cell death system as a potential cancer therapy (Kim et al (2016). Theranostics 6 357-368; Kwon et al, (2005). Mol. Ther 12, 5 824-834; Jung et al (2006). Biochem. Biophys. Res. Commun, 349, 556-563; Song et al (2009). Cancer Gene Ther 16, 113-125; Song et al (2006). FEBS Letters 580, 5033-5043; Won et al (2007). J. Biotechnol. 129, 614-619; Won et al (2012). J. Biotechnol. 158, 44-49). Herpes simplex virus thymidine kinase catalyses the conversion of the pro-drug ganciclovir into an active compound by phosphorylation, leading to chain termination during DNA replication and cell death (Duarte, S et al (2012). Cancer Letters 324, 160-170).

RNA trans-splicing has also been used to target HIV (Ingemarsdotter, C. K. et al (2017) Mol. Ther. Nucleic acids, 7, 140-154) and cancer-specific RNAs (Poddar et al (2018). Mol. Ther. Nucleic Acids, 11, 41-56; WO2017171654A1, US2015079678A1, WO2014068063A1).

### SUMMARY

The present inventors have developed an RNA trans-splicing molecule (RTM) that targets human endogenous retrovirus (HERV) pre-mRNA. This RTM may be useful in selectively killing cells that express HERV genes, for example cancer cells.

A first aspect of the invention provides an RNA trans-splicing molecule (RTM) comprising;
(i) a binding region specific for a HERV pre-mRNA,
(ii) a trans-splicing splice domain
(iii) a coding sequence for a suicide protein.

The binding region of the RTM binds to HERV pre-mRNA in a cell, such that the coding sequence is trans-spliced through the trans-splicing domain with the HERV pre-mRNA, resulting in a chimeric mRNA causing the suicide protein to be expressed in the cell.

Preferred RTMs may comprise the nucleic acid sequence of SEQ ID NO: 13, SEQ ID NO: 15 or a variant of either one of these.

A second aspect of the invention provides a nucleic acid encoding the RTM of the first aspect. Preferred nucleic acids may comprise the nucleotide sequence of SEQ ID NO: 14, SEQ ID NO: 16 or a variant of either one of these.

A third aspect of the invention provides an expression vector comprising a nucleic acid of the second aspect.

A fourth aspect of the invention provides viral particle comprising an RTM of the first aspect, a nucleic acid of the second aspect or an expression vector of the third aspect.

A fifth aspect of the invention provides an isolated cell comprising an RTM of the first aspect, a nucleic acid of the second aspect and/or an expression vector of the third aspect.

A sixth aspect of the invention provides a pharmaceutical composition comprising an RTM of the first aspect, a nucleic acid of the second aspect, an expression vector of the third aspect and/or a viral particle of the fourth aspect.

A seventh aspect of the invention provides a method of treatment of cancer comprising; administering an RTM of the first aspect, a nucleic acid of the second aspect, an expression vector of the third aspect, a viral particle of the fourth aspect and/or a pharmaceutical preparation of the sixth aspect to an individual in need thereof.

A method of the seventh aspect may further comprise administering to the individual a cytotoxic compound that is activated by the suicide protein. For example, an inactive pro-form may be administered to the individual and the suicide protein may convert the inactive pro-form into the active form of the cytotoxic compound in cells in which the suicide protein is expressed. The cytotoxic compound may be administered to the individual in a first treatment and one or more further treatments administered after the first treatment.

An eighth aspect of the invention provides an RTM of the first aspect, a nucleic acid of the second aspect, an expression vector of the third aspect, a viral particle of the fourth aspect and/or a pharmaceutical preparation of the sixth aspect for use in a method of treatment of cancer, for example a method of the seventh aspect; and the use of an RTM of the first aspect, a nucleic acid of the second aspect, an expression vector of the third aspect, a viral particle of the fourth aspect and/or a pharmaceutical preparation of the sixth aspect in the manufacture of a medicament for use in a method of treatment of cancer, for example a method of the seventh aspect.

The RTM, nucleic acid, expression vector, viral particle, and/or pharmaceutical preparation may be provided in combination with a cytotoxic compound that is activated by the suicide protein.

A ninth aspect of the invention relates a method of preventing cancer occurrence or recurrence in an individual undergoing cell therapy, the method comprising administering a population of cells according to the fifth aspect to an individual in need thereof.

The method may further comprise administering to the individual an inactive pro-form of a cytotoxic compound that is activated by the suicide protein, such that the cytotoxic compound is activated in cells in the population that have become or are becoming cancerous in the individual.

A tenth aspect of the invention relates to a method of killing a cell in vitro comprising;
contacting a cell with an RTM of the first aspect, a nucleic acid of the second aspect, an expression vector of the third aspect, a viral particle of the fourth aspect and/or a pharmaceutical preparation of the sixth aspect, such that the cell expresses the suicide protein; and,
contacting the cell with a cytotoxic compound that is activated by the suicide protein, such that the suicide protein activates the cytotoxic compound and kills the cell.

An eleventh aspect of the invention relates to a method of depleting HERV gene expressing cells in a population comprising;
contacting the population of cells with an RTM of the first aspect, a nucleic acid of the second aspect, an expression vector of the third aspect, a viral particle of the fourth aspect and/or a pharmaceutical preparation of the sixth aspect, such that HERV gene expressing cells in the population of cells express the suicide protein; and,
contacting the population with a cytotoxic compound that is activated by the suicide protein, such that the suicide protein activates the cytotoxic compound in HERV gene expressing cells in the population, thereby depleting HERV gene expressing cells in the population.

In preferred embodiments of the first to the eleventh aspects, the suicide protein may be HSV thymidine kinase and the cytotoxic compound may be ganciclovir.

Other aspects and embodiments of the invention are described in more detail below.

### BRIEF DESCRIPTION OF THE FIGURES

FIG. 1 shows the genome organisation of HERV-K class I and class II (top diagrams) with the gag, pro, pol and env open reading frames (ORFs) depicted. Pro and Pol are expressed through a −1 ribosomal frameshifting event. In HERV-K class I, there is a 292bp deletion within the env gene resulting in an alternative splicing event in HERV-K class I generating the Np9 RNA transcript whereas Rec is generated from HERV-K class II (middle diagrams). HERV-K env can be targeted for RNA trans-splicing with a RTM containing a binding domain complementary to the Rec/Np9 intron within env (bottom diagrams).

FIG. 2 shows the Np9 RNA trans-splicing target and binding domain sequence and alignments. 2A. RNA trans-splicing binding domain target sequence. The target sequence corresponds to nucleotide 6513-6556 of HERV-K HML-2-22q11.21, Genbank Sequence ID: JN675087.1 within the env open reading frame. 2B. Nucleotide alignment of selected target sequence with HERV-K HML-2-22q11.21, Genbank Sequence ID: JN675087.1 to confirm nucleotide positions 6513-6556. 2C. Reverse complementarity nucleotide sequence of target selected for optimisation of binding domain design (top). Optimised binding domain DNA sequence (middle). Optimised binding domain RNA sequence (bottom). Arrowed nucleotides depict wobble base substitution C to U for improvement of RNA structure, A to U mismatches, and A to G wobble base substitutions. 2D. Nucleotide alignment of binding domain sequence with target sequence HERV-K HML-2-22q11.21, Genbank Sequence ID: JN675087.1 showing sequence complementarity.

FIG. 3 shows RNA secondary structure predictions of binding domain sequence. 3A. Minimum free energy (MFE) (left) and centroid (right) RNA secondary structure predictions using RNA fold webserver. 3B. RNA secondary structure predictions of Np9-targeting RNA binding domains folded within the RNA trans-splicing cassette. RNA centroid fold (left) and MFE fold (right). The insets show the RNA binding domain region.

FIG. 4 shows the lentiviral RNA trans-splicing delivery system. 4A. Diagram showing a third generation lentiviral gene delivery system including the packaging plasmids (Gag/Pol, VSV-G, and Rev) and the gene transfer construct containing the RNA trans-splicing cassette depicted between XbaI and XhoI restriction enzyme sites. The RNA trans-splicing cassette is driven by an internal promoter, for example the thyroxine-binding globulin promoter (TBG) or cytomegalovirus promoter (CMV) or other tissue specific or non specific or system specific (e.g. hypoxia responsive) promoter. 4B. Detailed diagram of the RNA trans-splicing cassette shown in A. The TBG promoter is followed by the RNA binding domain sequence targeting HERV-K Np9. A spacer is included downstream of the binding domain for separation of the binding domain from the trans-splicing domain followed by a P2A cleavage site. The trans-gene Herpes simplex virus thymidine kinase (HSV-tk) lacks the first ATG translational initiation codon but the second and third ATGs (ATG⁴⁶ and ATG⁶⁰) are intact. A mini-intron is located further downstream. 4C. Optimised RNA trans-splicing cassette. The BbvCl restriction enzyme site and downstream nucleotides were mutated to disrupt a potential RNA splice acceptor site potentially causing RNA splicing in cis. CCTCAGCAGTG (BbvCl restriction enzyme site is underlined) was mutated to CCTC-GCGGTG. The second and the third ATGs encoding the AUG translational initiation sites were mutated from ATG to CTG and ATC for ATG⁴⁶ and ATG⁶⁰ respectively.

FIG. 5 shows HSV-tk protein expression in HEK 293T cells transfected with Np9-targeting RNA trans splicing constructs. 5A. Schematic of RNA trans-splicing constructs, HSV-tk-ts and HSV-tk-ts-opt, to induce RNA trans-splicing of HSV-tk pre-mRNA onto Np9 pre-mRNA. Both constructs are driven from a CMV promoter. HSV-tk-ts and its optimised version HSV-tk-ts-opt are described in FIG. 4C. 5B. Diagram showing HSV-tk mRNA. Arrows and green blocks indicate HSV-tk translational initiation sites. Four isoforms can be produced from wild type HSV-tk mRNA transcript, P1 from ATG1, P2 from a non-canonical ATG translational initiation site located between ATG1 and ATG46, P3 from ATG46, and P4 from ATG60. Of the different HSV-tk isoforms, it has been shown that P1 and P3 are catalytically active, P2 is inactive and P4 has very low activity (Ellison, A. R. & Bishop, J. O. (1996). Nucleic acids research 24 2073-2079). 5C. Predicted HSV-tk isoform protein expression of HSV-tk wild type transcript, HSV-tk-ts, and HSV-tk-ts-opt in both cells that express Np9 and cells that do not. Wild type HSV-tk transcript is expected to produce some degree of expression of P1, P2, P3, and P4. Both HSV-tk-ts and HSV-tk-ts-opt are expected to express P1 only in Np9 expressing cells. However, P3 and P4 expression is expected to be suppressed in HSVtk-ts-opt transfected cells. 5D. Protein lysate from HEK 293T cells analysed by western blot 72 hours after transfection with either of the RNA trans-splicing constructs, HSV-tk-ts or HSV-tk-ts opt, or controls, CMV-GFP or CMV-HSV-tk. HSV-tk antibody staining detected at both high and low exposures. 5E. Quantification of P1 expression in either HSV-tk-ts or HSV-tk-ts-opt transfected cells. P1 protein was normalized to vinculin protein in each lane (n=3). 5F. Quantification of P3 expression in either HSV-tk-ts or HSV-tk-ts-opt transfected cells. P1 protein was normalized to vinculin protein in each lane (n=3). For all graphs: Error bars indicate S.D.; ns=not significant; **P<0.01; unpaired t-tests.

FIG. 6 shows decreased cell viability in hepatocellular carcinoma cell lines (HCC) Hep3B and Huh7-mRFP after transduction with RNA trans-splicing lentiviral vectors targeting Np9 pre-mRNA. 6A. Hep3B cells (1×10^4 cells/well in a 96-well plate) were either untreated (ctrl), or transduced at an MOI (multiplicity of infection) of 1 with the negative control vector pSico, the trans-splicing vectors, 3′ER-HSVtk, 3′ER-HSVtk opt, or HSVtk (positive control). 3′ER-HSVtk and 3′ER-HSVtk opt are identical to HSV-tk-ts and HSV-tk-ts-opt respectively, shown in FIG. 5A, except for being driven from TBG promoters. One day post-transduction the cells were treated with a dose of 300 μM ganciclovir (GCV) followed by a second dose the next day. Four days after GCV treatment (five days post-transduction) cell viability was analysed by the MTT assay (Pannecouque, C. et al (2008) Nature protocols, 3, 427-434). 6B. HuH7-mRFP cells were transduced and treated with GCV as described in A. and cell viability analysed four days post-treatment. C. Hep3B cells were transduced as in A. and treated with GCV with two consecutive doses of 10 μM or 100 μM GCV at two and three days post-transduction followed by cell viability analysis by MTT assay three days later. Error bars indicate standard deviation (S.D.). Statistical analysis was performed with Student's t-test (two-tailed, assuming unequal variances), *P<0.05; **P<0.01; ***P<0.001, comparing each sample+GCV to without GCV for each sample.

FIG. 7 shows that RNA trans-splicing lentiviral vectors induce cellular death in HEK 293T cells and pancreatic cancer cell lines Panc-1, Aspc-1 and MIA PaCa-2. (7A-B) HEK 293T (7C-D) Panc-1 (7E-F) Aspc-1 (G-H) MIA PaCa-2 cells transduced with Lv-CMV-GFP, Lv-HSV-tk-ts, Lv-HSV-tk-ts-opt, or Lv-CMV-HSV-tk at an MOI of 1 or 2 and treated with 1000 μM GCV. Significant losses in cell viability in cells treated with RNA trans-splicing lentiviral vectors with CMV promoters was observed in almost all cell lines. Cell viability quantified using MTT assay. (n=3); Error bars indicate S.D.; *P<0.05; **P<0.01; ***P<0.001; ****P<0.0001; two-way ANOVA followed by Tukey's post-hoc multiple comparisons test.

### DETAILED DESCRIPTION

This invention relates to a ribonucleic acid (RNA) trans-splicing molecule (RTM) that comprises (i) a binding region specific for a HERV pre-mRNA, (ii) a trans-splicing domain and (iii) a sequence encoding a suicide protein. The RTM mediates the trans-splicing of a HERV pre-mRNA with the sequence that encodes the suicide protein, for example by 3′ exon replacement (3′ER) or 5′ exon replacement (5′ER). This leads to the expression of the suicide protein in cells that express the HERV pre-mRNA. Cells expressing the suicide protein may then be selectively killed by exposure to a cytotoxic compound that is activated by the suicide protein.

A ribonucleic acid (RNA) trans-splicing molecule (RTM) is a heterologous RNA molecule that is capable of inducing a trans-splicing event between an endogenous target pre-mRNA and the RTM, resulting in the generation of chimeric mRNA that comprises nucleotide sequences from both the target pre-RNA and the RTM. RTMs typically comprise a binding region, which defines the specificity for the target pre-mRNA, splicing elements that mediate trans-splicing and a coding sequence that replaces part of the target pre-mRNA. The design and use of RTMs for use in gene therapy has been reported (see for example, Wally et al. J Invest Dermatol 2012; 132: 1959-1966. Puttaraju et al Nat. Biotechnol. 1999; 17: 246-252). RTM described herein target HERV pre-mRNA. An RTM may be contacted with an HERV pre-mRNA, under conditions in which the coding sequence of the RTM is trans-spliced to the HERV pre-mRNA to form a chimeric mRNA molecule. This chimeric mRNA molecule may be further processed and expressed in the cell.

Features (i) to (iii) of the RTM may be arranged sequentially in a 5′ to 3′ direction in the order (i), (ii) (iii), for example for 3′ exon replacement; or in the order (iii), (ii), (i), for example for 5′ exon replacement (see for example Poddar et al (2018) Molecular therapy. Nucleic Acids 11, 41-56).

The RTMs described herein mediate trans-splicing with HERV precursor mRNA (pre-mRNA). Pre-mRNA is RNA that has been transcribed from a gene in the nucleus of a cell that has not yet been processed into mRNA. Pre-mRNA therefore contains introns and other features that are not present in mRNA. Pre-mRNA may also be referred as a primary transcript, or heterogeneous nuclear RNA (hnRNA).

A HERV pre-mRNA may comprise an unspliced or partially spliced transcript of a gene from a HERV provirus (a HERV gene). Suitable HERV pre-mRNA includes HERV-K pre-mRNA.

A human endogenous retrovirus (HERV) is an endogenous viral element or provirus that exists in the human genome. HERVs display a similar genomic organisation to exogenous retroviruses and are transmitted vertically in the germline through successive generations. The expression of genes from HERV proviruses is tightly regulated in normal cells but HERV-K genes may be dysregulated and over-expressed in cancers (see for example Hohn et al Frontiers in Oncology, 3, 246; Kassiotis, G. (2014) J. Immunol. 192, 1343-1349; Attig, J., et al Genome Res, 29, 1578-1590)

A HERV may be of any class or group, with a complete or an incomplete genome. For example, a HERV may be a class I HERV of any one of Groups 1 to 6, a class II HERV of any one of Groups 1 to 10 or a class III HERV. In some preferred embodiments, a HERV may be a Class II HERV, for example HERV-K. HERV-K is a class of human endogenous retroviruses (HERVs) within the human genome (see for example Bannert et al (2018) Front Microbiol 9, 178). HERV-2 may include HERV-K HML-2 proviruses, for example HERV-K HML-2 proviruses of Group 1 and Group 2.

Suitable HERV pre-mRNA may be transcribed from a HERV provirus gene, for example a gag, pro, pol or env gene, for example a HERV-K HML-2 gag, pro, pol or env gene.

In some preferred embodiments, the HERV provirus gene may be an env gene. For example, a suitable HERV pre-mRNA may be transcribed from a HERV env gene, for example a HERV-K env gene. A suitable HERV pre-mRNA is transcribed from a HERV-K HML-2 env gene. HERV-K HML-2 proviruses may be classified into two groups (Group 1 and Group 2). The env gene of HERV-K HML-2 Group 1 proviruses has a 291 bp deletion relative to the env gene of HERV-K HML-2 Group 2 proviruses. This deletion gives rise to alternative splicing in Group 1 and Group 2 proviruses to generate the Np9 protein in Group 1 proviruses and the Rec protein in Group 2 proviruses.

An RTM described herein may target both types of HERV-K HML-2 env gene transcript. For example, the HERV-K pre-mRNA may be (i) a HERV-K pre-mRNA that encodes Np9 and/or (ii) a HERV-K pre-mRNA that encodes Rec. For example, an RTM described herein may target both Np9 pre-mRNA expressed by HERV-K HML-2 Group 1 proviruses and Rec pre-mRNA expressed by HERV-K HML-2 Group 2 proviruses.

The binding region of the RTM specifically binds to HERV pre-mRNA. For example, the binding region may specifically bind to HERV pre-mRNA transcribed from the env gene. The binding of the binding region targets the RTM to the HERV pre-mRNA and allows the coding sequence encoding the suicide protein to be trans-spliced with the HERV pre-mRNA. In some preferred embodiments, the binding region may specifically bind to HERV-K pre-mRNA transcribed from the env gene, for example HERV-K Np9 or HERV-K Rec pre-mRNA. The binding of the binding region targets the RTM to the HERV-K pre-mRNA and allows the coding sequence encoding the suicide protein to be trans-spliced with the HERV-K pre-mRNA.

An RTM described herein may contain one or more binding regions i.e. it contains one contiguous sequence or multiple contiguous regions that hybridise to HERV pre-mRNA. Multiple binding regions may be useful for example in enhancing cell death (see for example Poddar et al (2018) supra). Preferably, an RTM described herein contains a single binding region i.e. it contains only one contiguous sequence that hybridises to HERV pre-mRNA.

The binding region may have an open structure. For example, it may lack self-complementary sequences and may comprise a sequence of unstructured nucleotides that are not paired or bound to other nucleotides in the RTM. The binding region may comprise, for example fewer than 25 consecutive unstructured nucleotides, for example 12 to 25 consecutive unstructured nucleotides.

Preferably, the binding region of the RTM specifically binds to an intron sequence of a HERV pre-mRNA, such as a HERV-K pre-mRNA. The binding region may specifically bind to an intron sequence close to a splice site in the HERV pre-mRNA. Suitable splice sites may be identified using standard sequence analysis tools (e.g. Neural Network Server within the Berkeley Drosophila Genome Project; CrypSkip software, Bioinformatics HUSAR server, German Cancer Research Centre). For 3′ exon replacement, the binding region may specifically bind to an intron sequence downstream (3′) of the splice site in the HERV pre-mRNA. For 5′ exon replacement, the binding region may specifically bind to an intron sequence upstream (5′) of the splice site in the HERV pre-mRNA.

The binding region may bind to a region that is identified as having high minimum free energy and high proportion of unstructured nucleotides relative to other intron sequences in the pre-mRNA. Suitable regions may be identified using standard sequence analysis tools (e.g. Foldanalyse, Bioinformatics HUSAR server, German Cancer Research Centre). For example, the binding region of an RTM targeting the HERV-K Env gene may bind within a region of the HERV-K Np9 or HERV-K Rec pre-mRNA that corresponds to SEQ ID NO: 1 (nucleotides 6513-6556 of HERV-K HML-2-22q11.21; Genbank ID: JN675087.1).

A suitable binding region may comprise the reverse complementary sequence of a sequence within the HERV pre-mRNA or a variant thereof. For example, a suitable binding region targeting an HERV-K gene may comprise the reverse complementary sequence of a sequence within the HERV-K pre-mRNA or a variant thereof. The binding region may comprise nucleotide sequence of SEQ ID NO: 3 (the reverse complementary sequence of SEQ ID NO: 1) or SEQ ID NO: 4 (the reverse complementary sequence of SEQ ID NO: 2) or may be a variant thereof. A suitable binding region may be fewer than 200 nucleotides in length, preferably fewer than 100 nucleotides in length.

In some preferred embodiments, the nucleotide sequence of the binding region may be modified to remove potential splice sites, prevent RNA editing and/or increase trans-splicing efficiency. For example, a binding region targeting HERV-K env pre-mRNA the may comprise nucleotide sequence of SEQ ID NO: 3 or a variant thereof, the nucleotide sequence having modifications relative to SEQ ID NO: 3 at one or more, preferably all, of positions 4, 19, 20, 32 and 34. For example, the nucleotide sequence may have a U at positions 4, 19, and 20, and G at positions 32 and 34. A suitable binding region may comprise a nucleotide sequence corresponding to SEQ ID NO: 3 with a C to U substitution at position 4; an A to U substitution at position 19; an A to U substitution at position 20; an A to G substitution at position 32; and an A to G substitution at position 34. For example, the binding region may comprise the nucleotide sequence of SEQ NO: 5 or a variant thereof.

A variant of a reference amino acid sequence or reference nucleotide sequence set out herein may comprise an amino acid sequence or a nucleotide sequence having at least 50%, at least 60%, at least 70%, at least 80%, at least 90%, at least 95%, or at least 98% sequence identity to the reference sequence. Particular amino acid sequence variants may differ from the reference sequence by insertion, addition, substitution or deletion of 1 amino acid, 2, 3, 4, 5, 6, 7, 8, 9, or 10 or more than 10 amino acids. Particular nucleotide sequence variants may differ from the reference sequence by insertion, addition, substitution or deletion of 1 nucleotide, 2, 3, 4, 5, 6, 7, 8, 9, or 10 or more than 10 nucleotides.

Sequence similarity and identity are commonly defined with reference to the algorithm GAP (Wisconsin Package, Accelerys, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Nucl. Acids Res. (1997) 25 3389-3402) may be used. Computerized implementations of these algorithms (GAP, BESTFIT, PASTA, and FASTA in the Wisconsin Genetics Software Package, Genetics Computer Group, 575 Science Dr., Madison, WI) are available and publicly available computer software may be used such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)), Genomequest™ software (Gene-IT, Worcester MA USA) and MAFFT (Katoh and Standley 2013, Molecular Biology and Evolution, 30(4) 772-780 software. When using such software, the default parameters, e.g. for gap penalty and extension penalty, are preferably used. A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Sequence comparison may be made over the full-length of the relevant sequence described herein.

An amino acid residue in a reference amino acid sequence may be altered or mutated by insertion, deletion or substitution, preferably substitution for a different amino acid residue, to produce a variant of the reference amino acid sequence. A nucleotide in a reference nucleotide sequence may be altered or mutated by insertion, deletion or substitution, preferably substitution for a different nucleotide, to produce a variant of the reference nucleotide sequence.

In some embodiments, a spacer may be located between the binding domain and the trans-splicing domain of an RTM described herein. The spacer may reduce or prevent interaction between the binding domain and the trans-splicing domain or the coding sequence. The spacer may comprise any nucleotide sequence that is not complementary to the binding domain region and does not hybridise or otherwise interact with the binding region, trans-splicing domain or the coding sequence. In some embodiments, the spacer may further comprise a stop codon to block translation of any unspliced RTM. A suitable spacer may comprise the nucleotide sequence of SEQ ID NO: 10 or may be a variant thereof.

In other embodiments, a RTM described herein may lack a spacer between the binding domain and the trans-splicing domain of the RTM.

After binding to HERV-K pre-mRNA through the binding region, the trans-splicing domain within the RTM undergoes spliceosome-mediated trans-splicing with the HERV pre-mRNA. Trans-splicing joins the nucleotide sequence encoding the suicide protein to trans-the HERV pre-mRNA. In a 3′ exon replacement, the coding sequence is located downstream (3′) of the trans-splicing domain and is spliced with the nucleotide sequence upstream (5′) of a donor splice site in the HERV pre-mRNA. In a 5′ exon replacement, the coding sequence is located upstream (5′) of the trans-splicing domain in the RTM and is spliced with the nucleotide sequence downstream (3′) of an acceptor splice site in the HERV pre-mRNA. Techniques for trans-splicing through 3′ and 5′ exon replacement are established in the art (see for example Poddar et al (2018) supra).

The trans-splicing of the RTM with the HERV pre-mRNA is mediated by the trans-splicing domain. The trans-splicing domain is a nucleotide sequence that comprises the motifs necessary to recruit the spliceosome and mediate trans-splicing with endogenous pre-mRNA. For example, the trans-splicing domain may comprise a splice site, at which the RTM is joined to the HERV pre-mRNA by trans-splicing.

The trans-splicing domain of an RTM suitable for use in 3′ exon replacement may comprise a splice acceptor site. The splice acceptor site may comprise an A-G dinucleotide sequence. For example, the splice acceptor site may comprise the sequence C-A-G-G. (e.g. AG). Suitable splice acceptor sites are well known in the art.

The trans-splicing domain of an RTM suitable for use in 3′ exon replacement may further comprise a polypyrimidine tract (PPT). The polypyrimidine tract may be located upstream of the splice site, for example 5 to 40 nucleotides upstream of the splice acceptor site in a 3′ exon replacement RTM. The polypyrimidine tract may comprise a sequence of 15-20 nucleotides that is rich in pyrimidines (C and U). Suitable PPTs include 5′-UUUUUUUCCCUUUUUUUCC-3′ and variants thereof. Other suitable PPTs are known in the art (see for example Wagner et al 2001 Mol Cell Biol 21(10):3281-3288; WO2017171654A1).

The trans-splicing domain of an RTM suitable for use in 3′ exon replacement may further comprise a branch point sequence. The branch point sequence may be located upstream of the PPT and may for example be 20 to 50 nucleotides upstream of the splice acceptor site. The branch point sequence may comprise the sequence YURAC or YNURAC, where R=purine, Y =pyrimidine and N=any nucleotide. Suitable branch point sequences include 5′-UACUAACA-3′ and are known in the art (see for example Gao et al Nucl Acid Res 2008 36(7) 2257-2267; US20060094675)

The trans-splicing domain of an RTM suitable for use in 3′ exon replacement may further comprise an intronic splice enhancer (ISE). The ISE may be located upstream of the branch point sequence. Suitable ISEs include 5′- GGG CCTGGGCCTG GG-3′ and are known in the art (see for example Wang et al Nat Struct. Mol. Biol. (2012) 19 (10) 1044-1052; McCarthy et al (1998) Hum Mol Genet 7 1491-1496; Yeo et al (2004) PNAS USA 101 15700-15705).

Trans-splicing domains suitable for use in 3′ exon replacement are well known in the art (see for example Poddar et al 2018 supra). For example, a suitable trans-splicing domain may comprise the nucleotide sequence of SEQ ID NO: 12 or may be a variant thereof.

A trans-splicing domain of an RTM suitable for use in 5′ exon replacement may comprise a splice donor site. The splice donor site may comprise a GU dinucleotide sequence. For example, the splice donor site may comprise the sequence 5′-CAG/GUAAGTAT-3′. Other suitable splice donor acceptor sites are well known in the art.

The trans-splicing domain of an RTM suitable for use in 5′ exon replacement may further comprise an intronic splice enhancer (ISE). The ISE may be located downstream of the splice donor sequence. Suitable ISEs are known in the art (see for example Wang et al Nat Struct. Mol. Biol. (2012) 19 (10) 1044-1052; McCarthy et al (1998) Hum Mol Genet 7 1491-1496; Yeo et al (2004) PNAS USA 101 15700-15705).

Trans-splicing domains suitable for use in 5′ exon replacement are well known in the art (see for example Poddar et al 2018 supra)

Preferably, an RTM described herein does not contain splice sites that interfere with the trans-splicing of the trans-splicing domain, for example by mediating cis-splicing. For example, a 3′ exon replacement RTM may lack splice sites upstream of the trans-splicing domain i.e. the trans-splicing domain is not downstream (3′) to a 5′ splice site in the 3′ER RTM. A 5′ exon replacement RTM may lack splice sites downstream of the trans-splicing domain i.e. the trans-splicing domain is not upstream (5′) to a 3′ splice site in the 5′ER RTM.

An RTM described herein may further comprise a separation element that allows the production of a suicide protein free of amino acids encoded by the HERV sequences in the chimeric mRNA. Suitable separation elements are well-known in the art (Poddar et al 2018 supra) and include stop codons and sequences encoding self-cleaving peptides, such as 2A peptides. The coding region of the suicide protein may be followed by a poly A tail such as a SV40 poly A (Poddar et al 2018).

For example, a 5′ ER RTM described herein may further comprise a stop codon, such as UAG, UAA or UGA, at the 3′ end of the sequence coding for the suicide protein. Translation of the chimeric mRNA after trans-splicing terminates at the stop codon, generating the suicide protein without additional amino acids encoded by the HERV sequences in the chimeric mRNA.

A 3′ ER RTM described herein may further comprise a self-cleaving peptide coding sequence, such as a 2A peptide coding sequence. The self-cleaving peptide coding sequence may be located between the trans-splicing domain and the coding sequence for the suicide protein, for example at the 5′ end of the coding sequence. The self-cleaving peptide causes cleavage of the nascent peptide chain during translation and separates the suicide protein from HERV amino acid sequences. Suitable 2A peptides may include T2A, P2A, E2A and F2A peptides (Poddar et al (2018) supra; Kim et al (2011) PLoS ONE 6, e18556.) and may comprise the amino acid sequence of SEQ ID NO: 11 or a variant thereof. A self-cleaving peptide coding sequence may comprise the nucleotide sequence of SEQ ID NO: 17 or a variant thereof.

A 5′ ER RTM described herein may further comprise ribozyme, such as a hammerhead ribozyme, either catalytically active or inactive, or a stabilizing RNA element. The ribozyme may be located downstream from the binding domain. The ribozyme, or stabilizing RNA element, may be followed by a spacer and a poly A tail. The ribozyme may remove nucleotide sequence downstream (3′) of the binding domain, such a polyA tail, from the RTM. Suitable ribozyme sequences, and stabilizing RNA elements are available in the art (Poddar et al (2018) supra).

An RTM described herein further comprises a coding sequence for a suicide protein. In a HERV gene expressing cell, such as cancer cell, the coding sequence is trans-spliced to the HERV pre-mRNA via the trans-splicing domain of the RTM, such that the suicide protein is expressed in the HERV gene expressing cell.

A suicide protein is a protein expressed in a cell that interacts with at least one other molecule to trigger or result in death of the cell. The coding sequence encoding the suicide protein may be referred to as a suicide gene. The coding sequence may comprise the complete coding sequence for a suicide protein. In a 3′ER RTM, the coding sequence may lack the 5′ translation initiation codon (start codon: AUG). This prevents the expression of the coding sequence in the absence of trans-splicing. In a 5′ER RTM, the coding sequence may include the 5′ translation initiation codon (start codon: AUG).

Preferably, the suicide protein is a prodrug activating enzyme that converts an inactive pro-form of a cytotoxic compound into an active form. Because the suicide protein generates the active cytotoxic compound from the inactive pro-form, cells that express the suicide protein are sensitive to exposure to the pro-form. Suitable suicide proteins for use as described herein are available in the art (see for example Malekshah et al (2016) Curr Pharmacol Reps 2 299-308) and include cytosine deaminase, which activates 5-fluorocytosine (5-FC); cytochrome P450, which activates ifosfamide (IFO) or cyclophosphamide; nitroreductase, which activates 5-[aziridin-1- yl]-2, 4-dinitrobenzamide; purine nucleoside phosphorylase, which activates fludarabine (ePNP; Secrist et al Nucleos. Nucleot. 1999, 18, 745-757; Krohne et al Hepatology (2001) 34(3):511-8) and thymidine kinase, which activates ganciclovir (GCV).

In some preferred embodiments, the suicide protein is Herpes simplex virus (HSV) thymidine kinase (tk). HSV-tk phosphorylates ganciclovir to produce the cytotoxic ganciclovir triphosphate. HSV thymidine kinase may comprise or consist of the sequence shown in SEQ ID NO: 6 (database accession number AF057310.1) or may be a variant thereof. A suitable coding sequence encoding HSV thymidine kinase may comprise a nucleotide sequence of SEQ NO: 7 or a variant thereof.

In an RTM suitable for 3′ exon replacement, the HSV thymidine kinase may lack an N terminal M residue at a position corresponding to position 1 of SEQ ID NO: 6. This may prevent expression of the suicide protein in the absence of a trans-splicing event. The coding sequence may lack a 5′ translation initiation site (AUG). In an RTM suitable for 5′ exon replacement, the HSV thymidine kinase may comprise an N terminal M residue at a position corresponding to position 1 of SEQ ID NO: 6.

In some embodiments, the coding sequence for the HSV thymidine kinase may comprise an exonic splice enhancer (ESE). Conveniently, alternative degenerative codons may be employed, so that the encoded amino acid sequence is not altered by the presence of the ESE. In some embodiments, the coding sequence may further comprise an intron, such as a 6-globin mini-intron. Suitable sequences are known in the art (Poddar et al 2018 supra).

Preferably, the coding sequence encoding the HSV thymidine kinase may be modified to reduce the expression of aberrant isoforms of thymidine kinase. This may be useful in reducing the production of active HSV thymidine kinase from sequences that are not trans-spliced to HERV-K pre-mRNA. For example, the HSV thymidine kinase may be modified to delete or replace M residues encoded by translation initiation codons. A suitable variant of the HSV thymidine kinase sequence of SEQ ID NO: 6 may lack one or more M residues encoded by translation initiation codons. For example, the M residues of the HSV thymidine kinase at positions corresponding to positions 1 and 46 of SEQ ID NO: 6, preferably positions 1, 46 and 60 of SEQ ID NO: 6 may be deleted or replaced by other residues. For example, the M residue of the HSV thymidine kinase at the position corresponding to position 1 of SEQ ID NO: 6 may be deleted. The M residue of the HSV thymidine kinase at the position corresponding to position 46 of SEQ ID NO: 6 may be replaced by a different residue, preferably an L residue. The M residue of the HSV thymidine kinase at the position corresponding to position 60 of SEQ ID NO: 6 may be replaced by a different residue, preferably an I residue. Preferably, the HSV thymidine kinase lacks functional translation initiation sites. A preferred HSV thymidine kinase encoded by the coding sequence may comprise the amino acid sequence of SEQ ID NO: 8 or a variant thereof.

A coding sequence for a modified HSV thymidine kinase may comprise an amino acid sequence that is a variant of SEQ ID NO: 7 with one or more codons that initiate translation modified. For example, one or more nucleotides of the translation initiation codon (ATG/AUG) at positions corresponding to positions 136-138 of SEQ ID NO: 7) may be replaced by other nucleotides, such that the codon ATG/AUG is disrupted or abolished. In some embodiments, the A at position 136 may be replaced with a different nucleotide. For example, a nucleotide sequence may comprise an A>C substitution at position 136. A suitable nucleotide sequence may lack a translation initiation codon at positions 136-138 and may not support the initiation of translation from these positions. One or more nucleotides of the translation initiation codon (ATG/AUG) at positions corresponding to positions 178-180 of SEQ ID NO: 7 may be replaced by other nucleotides, such that the translation initiation codon (ATG/AUG) is disrupted or abolished. In some embodiments, the G at position 180 may be replaced with a different nucleotide. For example, the nucleotide sequence may comprise a G>C substitution at position 180. A suitable nucleotide sequence may lack a translation initiation codon at positions 178-180 and may not support the initiation of translation from these positions.

In some preferred embodiments, the nucleotide sequence may lack translation initiation codons at positions 136-138 and positions 178-180. For example, the nucleotide sequence may comprise an A>C substitution at position 136 and a G>C substitution at position 180.

A preferred coding sequence for HSV thymidine kinase may comprise the nucleotide sequence of SEQ ID NO: 9 or a variant thereof.

A nucleic acid sequence encoding a modified HSV thymidine kinase as set out above may be generally useful as a suicide gene for RNA trans-splicing and is provided as an aspect of the invention. As described above, the modified HSV thymidine kinase may contain deletions or substitutions of residues M1 and M46, preferably Ml, M46 and M60. For example, the modified HSV thymidine kinase may contain an M46L mutation, preferably an M46L and an M601 mutation, as described above. The modified HSV thymidine kinase may comprise the sequence of SEQ ID NO: 8 or a variant thereof. The nucleic acid sequence may comprise the sequence of SEQ ID NO: 9 or a variant thereof. Nucleic acid constructs, such as RTMs and expression vectors, comprising the nucleic acid sequence and the use of such a nucleic acid in the production of a nucleic acid constructs, such as an RTM or an expression vector, are also provided as aspects of the invention. Constructs and nucleic acids may be useful in a range of trans-splicing applications, including 3′ exon replacement trans-splicing and ribozyme mediated trans-splicing.

A preferred RTM described herein may comprise the nucleotide sequence of SEQ ID NO: 13 or a variant thereof; or the nucleotide sequence of SEQ ID NO: 15 or a variant thereof.

An RTM as described above may be encoded by a nucleic acid, such as a DNA molecule. The nucleic acid molecule may be isolated. The nucleic acid may be partially or wholly synthetic.

A preferred RTM described herein may be encoded by a nucleic acid comprising the nucleotide sequence of SEQ ID NO: 14 or a variant thereof; or the nucleotide sequence of SEQ ID NO: 16 or a variant thereof.

The nucleic acid that encodes the RTM may be operably linked to one or more control elements or regulatory sequences capable of directing the expression of the RTM. Suitable control elements or regulatory sequences to drive the expression of heterologous nucleic acid coding sequences in mammalian cells, preferably human cells, are well-known in the art and include constitutive promoters, for example viral promoters such as CMV or SV40; and tissue specific promoters, for example promoters such as the human thyroxine binding globulin (TBG) promoter or system specific promoters such as hypoxia responsive promoters.

Tissue specific promoters may include cancer-specific promoters (i.e. promoters with activity specific to cancer cells) or promoters specific for the tissue in which cancer has occurred in an individual. Suitable promoters may include, for example, Cox-2 or Muc-1 promoters for pancreatic cancer.

Further provided are constructs in the form of plasmids, vectors (e.g. expression vectors), transcription or expression cassettes or other delivery systems which comprise an RTM described herein or a nucleic acid encoding the RTM described herein. For example, the nucleic acid encoding the RTM may be contained in an expression vector. Suitable expression vectors can be chosen or constructed, containing appropriate regulatory sequences, including promoter sequences, terminator fragments, polyadenylation sequences, enhancer sequences, marker genes and other sequences as appropriate. A vector may also comprise sequences, such as origins of replication, promoter regions and selectable markers, which allow for its selection, expression and replication in bacterial hosts such as E. coli.

Preferred vectors may be tropic for the cell type in which trans-splicing is required and may comprise suitable control and regulatory elements to enhance specific expression within that cell type. In some embodiments, the vector may be non-oncolytic, to avoid intrinsic deleterious cis-acting effects on splicing.

Vectors may be plasmids, viral e.g. phage, or phagemid, as appropriate. For further details see, for example, Molecular Cloning: a Laboratory Manual: 3rd edition, Russell et al., 2001, Cold Spring Harbor Laboratory Press. Many known techniques and protocols for manipulation of nucleic acid, for example in preparation of nucleic acid constructs, mutagenesis, sequencing, introduction of DNA into cells and gene expression, are described in detail in Current Protocols in Molecular Biology, Ausubel et al. eds. John Wiley & Sons, 1992.

In some preferred embodiments, the expression vector may be a viral vector, such as a lentivirus or adeno-associated virus (AAV) vector.

A viral expression vector is a recombinant nucleic acid that comprises viral sequences and a heterologous nucleic acid encoding the RTM to be expressed in a target cell. A viral expression vector may be packaged into a viral particle. A viral particle may comprise a viral expression vector encapsidated into a viral capsid. Suitable methods for packaging viral vectors into viral particles are well-established in the art.

It is possible to use a single viral expression vector that encodes all the viral components required for viral particle formation and function. Most often, however, multiple plasmid expression vectors or individual expression cassettes integrated stably into a host cell, such as a human embryonic kidney (HEK) 293 cell, are utilised to separate the various genetic components that generate the viral vector particles.

Expression cassettes encoding the one or more viral packaging and envelope proteins have been integrated stably into a mammalian cell. Transducing these cells with a viral expression vector described herein is sufficient to result in the production of viral particles without the addition of further expression vectors.

Alternatively, multiple expression vectors may be used. In some embodiments, mammalian cells may be transduced with one or more expression vectors encoding the viral packaging and envelope proteins that encode the viral packaging and envelope proteins necessary for particle formation. For example, a recombinant AAV vector may be prepared by co-transfecting a plasmid containing the heterologous nucleic acid flanked by two AAV inverted terminal repeat (ITR) regions, and a plasmid carrying the AAV encapsidation genes (rep and cap genes), into a cell line that is infected with a human helper virus (for example an adenovirus) or a cell line expressing isolated essential genes thereof). A recombinant lentiviral vector may be prepared by transfecting a packaging cell line, such as HEK293, with a transfer vector plasmid and two or more helper plasmids. The transfer plasmid contains the heterologous nucleic acid encoding the RTM, flanked by long terminal repeat (LTR) sequences, which facilitate integration of the transfer plasmid sequences into the host cell. The two or more helper plasmids may include one or more packaging plasmids which encode virion proteins, such as Gag, Pol, Tat, and Rev; and an envelope plasmid, which encodes an envelope protein, such as VSV-G; In some embodiments, two packaging plasmids may be employed, a first encoding Gag and Pol and a second encoding Rev. Following transfection with the transfer plasmid and helper plasmids, the packaging cell line generates infectious lentiviral particles that comprise the nucleic acid encoding the RTM. In some embodiments, a VSV-G-pseudotyped lentiviral vector may be produced in combination with the viral envelope glycoprotein G of the Vesicular stomatitis virus (VSV) to produce a pseudotyped lentivirus particle.

Recombinant cells, for example recombinant mammalian cells, that comprise a nucleic acid encoding an RTM described herein or a viral expression vector comprising a nucleic acid encoding an RTM described herein are provided. These may be useful for example in generating viral particles as described herein.

Viral particles may be harvested from the cell supernatant and stored and/or concentrated ready for use as described herein. Many known techniques and protocols for manipulation and transformation of nucleic acid, for example in preparation of nucleic acid constructs, introduction of DNA into cells and gene expression are described in detail in Protocols in Molecular Biology, Second Edition, Ausubel et al. eds. John Wiley & Sons, 1992. Reagents for generating viral vectors are available from commercial suppliers (e.g. Dharmacon). Suitable techniques for preparing viral vectors are well-known in the art (see for example, Dull, T., et al (1998). J. Virol. 72, 8463-8471; Merten et al (2016) Mol Ther Methods Clin Dev. 2016; 3: 16017).

While it is possible for an RTM, nucleic acid, expression vector or viral particle described herein to be used (e.g., administered) alone, it is often preferable to present it in the form of a pharmaceutical composition, which may comprise at least one component in addition to the RTM, nucleic acid, expression vector or viral particle. Thus pharmaceutical compositions may comprise, in addition to the RTM, nucleic acid, expression vector or viral particle, a pharmaceutically acceptable excipient, carrier, buffer, stabilizer or other materials well known to those skilled in the art.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation. Suitable carriers, excipients, etc. can be found in standard pharmaceutical texts, for example, Remington: The Science and Practice of Pharmacy , 23rd edition, Academic Press.

Pharmaceutical compositions and formulations may conveniently be presented in unit dosage form and may be prepared by any methods well known in the art of pharmacy. Such methods include the step of bringing into association the RTM, nucleic acid, expression vector or viral particle with the carrier which constitutes one or more accessory ingredients. In general, the compositions are prepared by uniformly and intimately bringing into association the active compound with liquid carriers.

A pharmaceutical composition comprising a RTM, nucleic acid, expression vector or viral particle may be administered alone or in combination with other treatments, either simultaneously or sequentially dependent upon the condition to be treated. For example, a pharmaceutical composition comprising an RTM, nucleic acid, expression vector or viral particle may be administered in combination with a cytotoxic agent that is activated by the suicide protein encoded by the coding sequence of the RTM.

In some embodiments, a pharmaceutical composition may further comprise a cytotoxic agent that is activated by the suicide protein. For example, the composition may comprise the pro-form of a cytotoxic agent that is converted into the active cytotoxic agent by the suicide protein.

The RTMs described herein which selectively kill cells expressing HERV genes and are thus useful in therapy, for example in the treatment of cancer. A RTM, nucleic acid, expression vector, viral particle or pharmaceutical composition as described herein may be used in a method of treatment of the human or animal body. The method of treatment may comprise administering the RTM, nucleic acid, expression vector, viral particle or pharmaceutical composition to an individual in need thereof. Therapeutic applications of RTMs are established in the art (see Hong et al (2020). Br Med Bull 136 4-20). Preferably the method is a method of treatment of cancer. A method of treatment of cancer as described herein may comprise administering an RTM, nucleic acid, vector, viral particle or pharmaceutical composition described herein to an individual in need thereof.

The method may further comprise administering to the individual a cytotoxic agent that is activated by the suicide protein encoded by the coding sequence of the RTM. For example, the pro-form of a cytotoxic agent that is converted into the active cytotoxic agent by the suicide protein may be administered to the individual.

Other aspects provide an RTM, nucleic acid, vector, viral particle or pharmaceutical composition described herein for use in a method of treatment of cancer and the use of an RTM, nucleic acid, vector, viral particle or pharmaceutical composition described herein in the manufacture of a medicament for use in method of treatment of cancer.

Cancers suitable for treatment as described herein may be characterised by the presence of one or more cancer cells that express an HERV gene. In some preferred embodiments, the one or more cancer cells may express a HERV-K gene, such as a HERV-K HML-2 env gene. For example, the cancer cells may express a HERV-K HML-2 Group 1 Np9 gene and/or a HERV-K HML-2 Group 1 Rec gene.

Suitable cancers may be familial or sporadic. Suitable cancers may be metastatic or non-metastatic.

For example, the cancer may be any type of solid or non-solid cancer or malignant lymphoma. The cancer may be selected from the group consisting of skin cancer (in particular melanoma), head and neck cancer, kidney cancer, sarcoma, germ cell cancer (such as teratocarcinoma), liver cancer (such as hepatocellular carcinoma), lymphoma (such as Hodgkin's or non-Hodgkin's lymphoma), leukaemia, such as acute myelogenous or myeloid leukemia (AML), acute lymphoblastic leukemia (ALL), chronic lymphatic leukemia (CML), chronic myelogenous or myeloid leukemia (CML), hairy cell leukemia (HCL), T-cell prolymphocytic leukemia (P-TLL), large granular lymphocytic leukemia, adult T-cell leukaemia, skin cancer, bladder cancer, breast cancer, uterine cancer, ovarian cancer, prostate cancer, lung cancer, colorectal cancer, cervical cancer, oesophageal cancer, pancreatic cancer (such as pancreatic ductal adenocarcinoma), stomach cancer, and cerebral cancer, preferably ovarian cancer, colon cancer, breast cancer, melanoma, leukaemia, testicular cancer, and prostate cancer.

In some preferred embodiments, the cancer may be a liver cancer, such as hepatocellular carcinoma, or a pancreatic cancer, such as pancreatic ductal adenocarcinoma.

In some embodiments, a cancer for treatment as described herein may have been previously identified as expressing a HERV gene or expressing a HERV gene above a threshold value. In other embodiments, a method may comprise identifying a cancer as expressing a HERV gene or expressing a HERV gene above a threshold value and treating the cancer as described herein. A method of selecting an individual with cancer who is likely to respond to treatment with an RTM as described herein may comprise;
determining the expression of a HERV gene in a sample obtained from the individual,
expression of the HERV gene being indicative that the individual is likely to respond to the treatment

The sample may be a sample of cancer cells or a sample of blood or other biological fluid comprising cell-free nucleic acid from cancer cells in the individual.

Suitable techniques for determining the expression of a HERV gene are well known in the art.

An individual suitable for treatment as described above may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human.

In some preferred embodiments, the individual is a human. In other preferred embodiments, non-human mammals, especially mammals that are conventionally used as models for demonstrating therapeutic efficacy in humans (e.g. murine, primate, porcine, canine, or lagomorph animals) may be employed.

Administration is normally in a “therapeutically effective amount” or “prophylactically effective amount”, this being sufficient to show benefit to a patient. Such benefit may be at least amelioration of at least one symptom. The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners.

A RTM, nucleic acid, expression vector, viral particle or pharmaceutical composition as described herein may be administered alone or in combination with other treatments, either simultaneously or sequentially dependent upon the circumstances of the individual to be treated.

Prescription of treatment, e.g. decisions on dosage etc, is within the responsibility of general practitioners and other medical doctors and may depend on the severity of the symptoms and/or progression of a disease being treated. Appropriate doses of therapeutic polypeptides are well known in the art (Ledermann J. A. et al. (1991) Int. J. Cancer 47: 659-664; Bagshawe K. D. et al. (1991) Antibody, Immunoconjugates and Radiopharmaceuticals 4: 915-922). Specific dosages may be indicated herein or in the Physician's Desk Reference (2003) as appropriate for the type of medicament being administered may be used. A therapeutically effective amount or suitable dose of a RTM, nucleic acid, expression vector, viral particle or pharmaceutical composition as described herein may be determined by comparing its in vitro activity and in vivo activity in an animal model. Methods for extrapolation of effective dosages in mice and other test animals to humans are known. The precise dose will depend upon a number of factors, including whether the RTM, nucleic acid, expression vector, viral particle or pharmaceutical composition as described herein is for prevention or for treatment, the size and location of the area to be treated, and the precise nature of the RTM.

Treatments may be repeated at daily, twice-weekly, weekly or monthly intervals, at the discretion of the physician. The treatment schedule for an individual may be dependent on the immunological, pharmocokinetic and pharmacodynamic properties of the RTM, nucleic acid, expression vector, viral particle or pharmaceutical composition, the route of administration and the nature of the condition being treated.

Following treatment with the RTM, nucleic acid, expression vector, viral particle or pharmaceutical composition treatment with the activatable cytotoxic agent may be periodic, and the period between administrations may be about one week or more, e.g. about two weeks or more, about three weeks or more, about four weeks or more, about once a month or more, about five weeks or more, or about six weeks or more. For example, treatment may be every two to four weeks or every four to eight weeks. This may be useful, for example in selectively killing cells that become cancerous after the initial treatment and may be useful in preventing or reducing the risk of relapse.

Treatment with the RTM, nucleic acid, expression vector, viral particle or pharmaceutical composition and/or the pro-form of the cytotoxic agent may be given before, and/or after surgery, and/or may be administered or applied directly at the anatomical site of trauma, surgical treatment or invasive procedure. Suitable formulations and routes of administration are described above.

The RTM, nucleic acid, expression vector, viral particle or pharmaceutical composition may be administered in combination with the cytotoxic agent that is activated by the suicide protein encoded by the coding sequence of the RTM. Pro-forms of cytotoxic agents that can be converted into the active agent by the suicide protein may be readily administered to patients using standard medical approaches. Methods known in the field may also be used to determine the most appropriate dose and route for the administration of the pro-form. For example, ganciclovir may be administered systemically (e.g. orally or parenterally) in a dose of about 1-20 mg/day/kg body weight; acyclovir may be administered in a dose of about 1-100 mg/day/kg body weight, and FIAU may be administered in a dose of about 1-50 mg/day/kg body weight.

As used herein, the terms “cancer,” “neoplasm,” and “tumour” are used interchangeably and, in either the singular or plural form, refer to cells that have undergone a malignant transformation that makes them pathological to the host organism.

Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumour, a “clinically detectable” tumour is one that is detectable on the basis of tumour mass; e.g., by procedures such as computed tomography (CT) scan, magnetic resonance imaging (MRI), X-ray, ultrasound or palpation on physical examination.

Cancer cells that express a HERV gene and are selectively targeted by the RTM described herein are more susceptible and sensitive than other cells to treatment with the cytotoxic agent because they are exposed to the active cytotoxic agent that results from conversion of the pro-form by the suicide protein.

In addition, activated cytotoxic agent can passively diffuse to neighbouring cancer cells to further enhance cancer cell death. This “bystander effect” increases the efficacy of the treatment.

An individual with cancer may display at least one identifiable sign, symptom, or laboratory finding that is sufficient to make a diagnosis of cancer in accordance with clinical standards known in the art. Examples of such clinical standards can be found in textbooks of medicine such as Harrison's Principles of Internal Medicine, 15th Ed., Fauci A S et al., eds., McGraw-Hill, New York, 2001. In some instances, a diagnosis of a cancer in an individual may include identification of a particular cell type (e.g. a cancer cell) in a sample of a body fluid or tissue obtained from the individual.

Treatment may be any treatment and therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumour volume or morphology (for example, as determined using computed tomographic (CT), sonography, or other imaging method), a delayed tumour growth, or a destruction of tumour vasculature. Administration of RTM, nucleic acid, vector, viral particle or pharmaceutical composition described herein may improve the capacity of the individual to resist cancer growth, in particular growth of a cancer already present in the subject and/or decrease the propensity for cancer growth in the individual.

The RTMs described herein which selectively kill cells expressing HERV genes and may also be useful in preventing cancer occurrence or recurrence in individuals undergoing cell therapy i.e. individuals receiving autologous or allogeneic cells for the treatment of a disease condition, such as cancer. Cells in an individual may express a HERV gene when they become or are in the process of becoming cancerous. These cells may be killed as described herein before a cancer condition develops in the individual. For example, a method of preventing cancer occurrence or recurrence in an individual having a population of cells administered thereto, the method comprising administering to the individual in need thereof said population of cells, wherein the cells comprise a RTM, nucleic acid, expression vector or viral particle as described herein. A cytotoxic compound that is activated by the suicide protein may be subsequently administered to the individual, such that activation of the cytotoxic compound occurs in cells of the population have become or are becoming cancerous in the individual.

Suitable cells for use in cell therapy are well known in the art and may include for example, haematopoietic cells, such as T cells, haematopoietic stem cells and bone marrow cells.

An RTM, nucleic acid, vector, viral particle or pharmaceutical composition described herein may also be useful in delivering nucleic acid encoding a suicide protein in vitro or ex vivo, for example for use in killing HERV gene expressing cells. A method of delivering nucleic acid encoding a suicide protein into a cell in vitro may comprise;
introducing an RTM, nucleic acid, vector or viral particle as described herein into the cell, such that expression of a HERV gene in the cell causes the suicide protein be expressed.

The cell may express a HERV gene or may undergo a transformation event that causes expression of a HERV gene. A RTM in a HERV gene expressing cell may be trans-spliced to a HERV pre-mRNA to form a chimeric mRNA molecule comprising the nucleotide sequence encoding the suicide protein. The chimeric mRNA may be further processed and translated, such that the suicide protein is expressed in the cell. The cell may be contacted with a cytotoxic agent that is activated by the suicide protein, such that the cytotoxic agent is activated by the suicide protein and kills the cell.

The methods described here may be useful in killing cells in vitro or ex vivo. A method of killing a cell in vitro or ex vivo may comprise;
introducing an RTM, nucleic acid, vector or viral particle as described herein into a cell,
such that expression of a HERV gene in the cell causes expression of the suicide protein, and
contacting the cell with a cytotoxic agent that is activated by the suicide protein,
such that the cytotoxic agent is activated by the suicide protein following expression of a HERV gene in the cell.

The methods described herein may be useful in depleting HERV gene expressing cells in a population of cells. For example, a method of depleting HERV gene expressing cells in a population comprising;
introducing an RTM, nucleic acid, vector or viral particle as described herein into a population of cells, such that the suicide protein is expressed in HERV gene expressing cells in the population of cells; and,
contacting the population with a cytotoxic compound that is activated by the suicide protein, such that the cytotoxic compound is activated in HERV gene expressing cells in the population, thereby depleting HERV gene expressing cells in the population.

HERV gene expressing cells may include cancer cells or pre-cancerous cells. The methods described herein may be useful in depleting or removing cancer cells or pre-cancerous cells from a population of cells.

The population of cells may be a sample of cells derived or obtained from an individual or cultured cells descended therefrom. Suitable cells include mammalian, preferably human cells. For example, suitable cells may include cells for use in cell therapy, for example haematopoietic cells, such as T cells, haematopoietic stem cells and bone marrow cells.

The RTM, nucleic acid, vector or viral particle may be introduced into the cell in vitro or ex vivo by any convenient technique, such as transfection, lipofection, transduction, electroporation, nucleofection or transformation.

Other aspects and embodiments of the invention provide the aspects and embodiments described above with the term “comprising” replaced by the term “consisting of”and the aspects and embodiments described above with the term “comprising” replaced by the term “consisting essentially of”.

The term “downstream” as used herein refers to the 5′ to 3′ direction in a nucleic acid described herein and the term “upstream” as used herein refers to the 3′ to 5′ direction in a nucleic acid described herein

Reference to a nucleotide sequence as set out herein encompasses a DNA molecule with the specified sequence, and encompasses a RNA molecule with the specified sequence in which U is substituted for T, unless context requires otherwise.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

### Experimental

The described RNA trans-splicing invention targets a large number of Np9 encoding class I proviruses, or Rec from class II proviruses, by targeting the env intron using a universal binding domain sequence. As a result of a RNA trans-splicing reaction, a HSV-tk gene that we have optimised to eliminate aberrant HSV-tk isoform production and consequent undesired off-target effects, is expressed.

### Materials and Methods

### Generation of RNA Trans-Splicing Binding Domain

An Np9-targeting RNA trans-splicing binding domain was generated from the following endogenous retroviral sequence, Homo sapiens isolate HML-2_22q11.21 endogenous virus HERV-K, complete sequence (GenBank: JN675087.1) based on previously described methods (Ingemarsdotter, C. K. et al (2017) Mol. Ther. Nucleic acids, 7, 140-154). Briefly, minimum free energy (MFE) calculations of the reverse complement of the target sequence was performed using the Foldanalyze software within the

Bioinformatics HUSAR server (German Cancer Research Centre) with a window size of 50 nucleotides and a stepsize of 1. The HML-2_22q11.21 sequence (GenBank: JN675087.1) was subjected to Splice Site Predictions using the Neural Network Server within the Berkeley Drosophila Genome Project, and the CrypSkip software within the Bioinformatics HUSAR server. Potential binding domain sequences were selected in Foldanalyze for further RNA secondary structure predictions based on high minimum free energy and unpaired nucleotides in proximity to, and downstream of, the Np9 splice donor site (Armbruester, V. et al (2002). Clin. Cancer. Res. 8, 1800-1807).

RNA secondary structures of selected potential binding domain regions were predicted using the webservers Mfold (Zuker, M., (2003) Nucleic acids research, 31, 3406-3415 and RNA fold. Selected binding domain regions were refolded within the RNA trans-splicing cassette backbone to exclude long-distance interactions of the binding domain. The binding domain structure was optimised by introduction of wobble base pairing (C to U or A to G modifications) to reduce duplex formation. Two mismatch nucleotides were introduced as previously described (Ingemarsdotter et al (2017)) to prevent effects triggered by long-double stranded RNA (Chalupnikova, K. et al (2013). Methods Mol. Biol. 942, 291-314).

In addition, the BbvCl restriction enzyme site and adjacent nucleotides flanking the binding domain in the RNA trans-splicing cassette was altered from CCTCAGCAGTG to CCTC-GCGGTG to disrupt a potential splice acceptor site as identified by splice site predictions using the Neural Network Server within the Berkeley Drosophila Genome Project. After selection of a promising binding domain structure, and optimisation of the binding domain structure, the RNA trans-splicing binding domain and flanking region in the RNA trans-splicing cassette sequence (between NheI to MluI restriction enzyme sites) were analysed for potential splice donor and splice acceptor sites by splice site prediction using the Neural Network Server within the Berkeley Drosophila Genome Project. After any mutations had been introduced, to avoid potential splice donor and splice acceptor site, the binding domain and flanking region (NheI to MluI) was reanalysed by splice site prediction by Neural Network Server within the Berkeley Drosophila Genome Project to confirm that introduced mutations did not introduce novel splice donor and splice acceptor sites.

### Generation of HSV-tk Translational Initiation Mutants

The Np9-targeting binding domain and flanking sequences were synthesised by GeneArt synthesis with flanking NheI and MluI restriction enzyme sites (Life Technologies). This insert was cloned into the BD100-HSVtk and BD100-HSVtk opt2 pVAX-1 backbones driven from the CMV promoter using NheI and MluI restriction enzyme (Thermo Scientific) digestion and ligation to replace the HIV-targeting binding domain BD100 region (Ingemarsdotter et al (2017) Mol. Ther. Nucleic acids, 7, 140-154). The BD100 and BD72 binding domain regions correspond to binding domain regions BD1-D4 and BD2-D4 respectively, as described in Ingemarsdotter et al. The HSV-tk translational initiation mutations at HSV-tk ATG⁴⁶ and ATG⁶⁰ were generated in the HIV-targeting RNA trans-splicing construct CkRhsp-BD72-pVAX-1 by site-directed mutagenesis using QuickChange II XL site-directed mutagenesis kit (Agilent Technologies) according to the manufacturer's instructions with 10 ng of plasmid CkRhsp-BD72-pVAX-1 as template and site-directed mutagenesis primers;

forward:
5′-GAAACTGCCCACGCTACTGCGGGTTTATATAGACGGTCCCCACGGG
ATCGGG-3′
reverse:
5′-CCCGATCCCGTGGGGACCGTCTATATAAACCCGCAGTAGCGTGGGC
AGTTTC-3′.

Mutagenesis of the second ATG, ATG⁴⁶ was confirmed by sequencing and 10 ng of the resulting plasmid DNA was used a template in a second round of mutagenic PCR to mutate the third HSV-tk ATG, ATG⁶⁰, with mutagenesis primers

tktr 3 mut forward;
5′-ACGGTCCCCACGGGATCGGGAAAACCACCAC-3′,
and
tktr 3 mut rev;
5′-GTGGTGGTTTTCCCGATCCCGTGGGGACCGT-3′

using Phusion HF DNA polymerase with 5× Phusion HF reaction buffer (New England Biolabs), 125 ng forward and reverse mutagenic primers, 1μl dNTP mix (Agilent Technologies), 3 μl Quicksolution (Agilent Technologies). Mutagenic PCR conditions: −95° C. for 1min followed by 95° C. for 50sec, 60° C. for 50 sec, 68° C. for 4 min during 18 cycles, followed by 68° C. for 7 min. The PCR products were digested for 1 h at 37° C. with Dpnl and transformed into XL10-Gold ultracompetent cells (Agilent Technologies). The resulting HSV-tk opt domain was subcloned into the ChkRhsp-BD100 opt 1-pVAX-1 backbone using PstI and MluI restriction digestion, ligation and subcloning, to generate ChkRhsp-BD100 opt 2-pVAX-1. The ChkRhsp promoter was replaced with a CMV promoter in the ChkRhsp-BD100 HSV-tk opt 2-pVAX-1 and BD100-HSV-tk-pVAX-1 backbones using SpeI and NheI restriction enzyme digestion, ligation and subcloning.

The Np9-targeting binding domain and flanking sequences were synthesised by GeneArt synthesis with flanking NheI and MluI restriction enzyme sites (Life Technologies). This insert was cloned into the CMV-BD100-HSVtk and CMV-BD100-HSVtk opt2 pVAX-1 backbones using NheI and MluI restriction enzyme (Thermo Scientific) digestion and ligation to replace the HIV-targeting binding domain BD100 region described in Ingemarsdotter et al (2017) with the Np9-targeting binding domain.

Generation of RNA Trans-Splicing pVAX-1 Shuttle Plasmid

To facilitate subcloning of RNA trans-splicing cassettes from pVAX-1 into a third generation lentiviral gene transfer plasmid pSico (Addgene, ref:11578) restriction enzyme sites for XbaI and XhoI were introduced flanking the HIV-targeting RNA trans-splicing cassette, XbaI at the 5′end and XhoI at the 3′end upstream of the poly-A tail within the RNA trans-splicing cassette in the CkRhsp-BD100-opt2 pVAX-1 backbone (Ingemarsdotter et al (2017)) using QuickChange XL site-directed mutagenesis kit (Agilent Technologies) according to the manufacturer's instructions with 10 ng of plasmid CkRhsp-BD100-opt2-pVAX-1 as template.

In the first mutagenic PCR, an XbaI restriction enzyme site was introduced at the 5′ end of the RNA trans-splicing cassette using the following mutagenic primers;

5′XbaI forward;
5′-GACATTGATTATTGTCTAGAACTAGTTGAGCCCCACG-3′.
5′XbaI reverse;
5′-CGTGGGGCTCAACTAGTTCTAGACAATAATCAATGTC-3′.

The PCR product was digested with DpnI for 1 h at 37° C. degrees and transformed into XL-10 Gold ultracompetent cells (Agilent Technologies). Positive clones were confirmed by sequencing and used as a template in a second round of mutagenic PCR to introduce an XhoI site at the 3′end within the RNA trans-splicing cassette upstream of the polyA tail. The primers used for the second mutagenic PCR were as follows;

3′XhoI forward;
5′-GGGAGGCGAACTGACTCGAGAACTTGTTTATTGC-3′
and
3′XhoI reverse;
5′-GCAATAAACAAGTTCTCGAGTCAGTTCGCCTCCC-3′.

The Np9-targeting trans-splicing domain was cloned into the pVAX-1 shuttle 5′XbaI-BD100-opt1 and 5′XbaI-BD100-opt2 plasmids using SpeI and PvuI restriction enzyme digestion and ligation. The resulting plasmids, HSVtk-ts-pVAX shuttle and HSVtk-ts-opt-pVAX shuttle, were digested with XbaI, XhoI and NsbI to isolate the Np9-targeting RNA trans-splicing cassettes between XbaI and XhoI followed by ligation into the pSico lentiviral backbone at the XbaI and XhoI restriction enzyme sites.

### Amplification of Thyroxine-Binding Globulin Promoter

The thyroxine binding globulin promoter (TBG) was amplified by PCR from plasmid
pAAV.TBG.PI.Null.bGH (Addgene plasmid: 105536) using the following forward primer with a flanking BcuI restriction enzyme site
TG BcuI forward, 5′-GCACTAGTTGCATGTATAATTTCTACAG-3′ and reverse primer with flanking NheI restriction enzyme site
TG NheI reverse 5′-CGGCTAGCTTATAGCATGTCCTGTATTG-3′.

20 ng of plasmid pAAV.TBG.PI.Null.bGH (Addgene plasmid: 105536) was used as a template in a PCR reaction containing 1× GoTaq Reaction buffer (Promega), 500 nM forward and 500 nM reverse primer, 200 μM PCR nucleotide mix (Promega), GoTaq G2 DNA polymerase 1.25 U in a final reaction volume 50 μl. PCR cycling conditions were; 95° C. for 2 min followed by 95° C. for 30 sec, 55° C. for 30 sec, 72° C. for 30 sec during 35 cycles, followed by 72° C. for 5 min and a 4° C. holding step. The TBG promoter was cloned into the -HSVtk-ts-pVAX shuttle and HSVtk-ts-opt-pVAX shuttle plasmids to replace the CMV promoter using BcuI and NheI double digestion and cloning. The resulting TBG-HSVtk-ts-pVAX shuttle and TBG-HSVtk-ts-opt-pVAX shuttle were digested with XbaI, XhoI and NsbI to isolate the Np9-targeting RNA trans-splicing cassettes between XbaI and XhoI followed by ligation into the pSico lentiviral backbone.

### Cell Culture

Hep3B (ATCC), HuH-7 (JCRB Cell Bank) engineered to express mRFP (HuH-7-mRFP), HEK 293T, MIA PaCa-2 (PHE (ECACC), and Panc-1 cells (PHE (ECACC), were cultured in Dulbecco's Modified Eagle Media (DMEM; Sigma) supplemented with 10% fetal bovine serum (FBS; Gibco) and 1% penicillin streptomycin (Gibco). Aspc-1 cells (ATCC) were cultured in RPMI media (Sigma) supplemented with 10% FBS and 1% penicillin-streptomycin. All cells were maintained at 37° C. in a humidified 5% CO2 atmosphere.

### Western Blot

HEK 293T cells were seeded in a 24 well plate at a density of 1×10⁵ cells/well in 1 mL/well of supplemented DMEM. 24 hours later, cells were transfected with 0.5 μg plasmid (pSico [CMV-GFP], pS-HSV-tk-ts, pS-HSV-tk-ts-opt, pS-CMV-HSV-tk). Three days post-transfection, cells were washed with phosphate-buffered saline (PBS; Sigma), harvested using 1× cell culture lysis reagent (Promega), and incubated for 15 minutes at room temperature. Lysates were then spun at 12000 rpm for 2 minutes. Supernatants were collected and treated with protease inhibitor cocktail (Halt). Protein concentrations were quantified using a bovine serum albumin (BSA; Sigma Aldrich) standard and Bradford dye reagent (Bio-Rad). Subsequently, 6-mercaptoethanol-containing loading dye was added to 5 μg of protein and boiled at 95° C. for 5 minutes. Prepared samples were electrophoresed on a 10% SDS-PAGE gel, and transferred to nitrocellulose paper using transfer buffer (25 mM Tris base, 150 mM glycine, 10% ethanol) at 100V for 1 hour. Membranes were blocked for 1 hour at room temperature using blocking buffer (4% BSA, 0.05% Tween in PBS), and incubated overnight at 4oC with goat anti-HSV-1 Thymidine Kinase antibody vN-20 (1:5000; Santa Cruz Biotechnology sc28037) in staining solution (1.5% BSA, 0.05% Tween in PBS). Next, membranes were washed three times with PBS-0.05% Tween, incubated with rabbit anti-goat HRP conjugated antibody (1:2000 Dako P0160) in staining solution for 1 hr at room temperature, and finally washed three more times with PBS-0.05% Tween. Stained membranes were developed using ECL western blot substrate (Promega) and visualized with high contrast blue sensitive X-ray film. Subsequently, blots were washed twice in PBS, incubated in stripping buffer (Thermo Scientific) for 15 minutes, and washed with PBS three more times. Gels were re-stained using rabbit anti-vinculin antibody (1:5000; Invitrogen 700062) and goat anti-rabbit HRP-conjugated secondary antibody (1:5000; Invitrogen 656120) and visualized using ECL western blot substrate. Western blot quantification was completed using Fiji software (https://imagej.net/Fiji).

### MTT Cell Viability Assay

Cell viability was evaluated based on previously established methods (Pannecouque et al (2008). Nature protocols, 3, 427-434). Briefly, on day 1, Hep3B, HuH7-mRFP, HEK 293T, MIA PaCa-2, Panc-1, and Aspc-1 cells were seeded in a 96 well plate at a density of 2×10⁴ cells/well in 100 μl media/well. On day 2, HEK 293T, MIA PaCa-2, Panc-1, and Aspc-1 cells were transduced with lentivirus (Lv-CMV-GFP, Lv-HSV-tk-ts, Lv-HSV-tk-ts-opt, Lv-CMV-HSV-tk) at an MOI of either 1 or 2. On day 4, cells were treated with 1000 μM of GCV (Sigma). Hep3B or HuH7-mRFP cells were transduced at an MOI of 1 with lentiviral vectors; pSico (negative control), the trans-splicing vectors, 3′ER-HSVtk, 3′ER-HSVtk opt, or HSVtk (positive control). Hep3B and HuH7-mRFP cells were treated with GCV at various concentrations on day 3 and 4. Finally, on day 8, the Tetrazolium dye MTT (3-[4,5-Dimethyl-2-thiazol-2-yl]-2,5-diphenyl-tetrazolium bromide) (Sigma) was added to media in wells to a final concentration of 10% (0.5 mg/ml) and incubated for 2-2.5 hours at 3TC. Media was then removed and replaced with acidified isopropanol triton X-100 solution (0.04N HCl, 6% triton X-100) and incubated at room temperature for 15 min. Absorbance was measured at both 595 nm and 655 nm on an iMark Microplate Absorbance Reader (Bio-Rad). Background fluorescence measurements read at 655 nm were subtracted from those at 595 nm. Cell viability was calculated by setting the average fluorescence of all untreated wells on each plate to 100%.

### Lentiviral Vector Production

Lentiviral vectors were produced as previously described (Dull et al (1998) Journal of Virology, 72, 8463-8471) with additional Benzonase treatment to remove plasmid carry-over Briefly, HEK 293T cells were seeded onto a 10-cm dish at density of 5×10⁶ cells/plate. 24 hours later, cells were transfected with 10 μg transfer plasmid (pSico, pS-HSV-tk-ts, pS-HSV-tk-ts-opt, pS-CMV-HSV-tk) or (3′ER-HSVtk, 3′ER-HSVtk opt, or TBG-HSVtk (positive control) driven by the TBG promoter in the pSico plasmid backbone)), 6.5 μg pMDLg/pRRE (Gag and Pol expressing packaging plasmid), 2.5 μg pRSV-REV (Rev expressing packaging plasmid), and 3.5 μg VSV-G envelope expressing plasmid) based on previous methods (Dull et al (1998) supra), using TransIT-LT1 transfection reagent (Mirus). At 24 hours post-transfection, media was removed and replaced with fresh DMEM. At 48 hours post-transfection, media was collected and centrifuged at 3000 rpm for 10 minutes to remove cell debris. Supernatant was collected and treated with 50 U/mL Benzonase (Sigma) and incubated for 30 minutes at 37° C. To remove remaining debris, supernatant was subsequently passed through a 0.45 μm surfactant-free cellulose acetate syringe filter (Sartorius Minisart) into appropriate ultracentrifuge tubes, weighed, and centrifuged at 20,000×g for 90 minutes at 4° C. Supernatant was removed from tubes, and lentiviral pellets were resuspended in 500 μl PBS.

Infectivity Titre Calculation Using qPCR

Briefly, HEK 293T cells were seeded in a 12 well plate at 2×10⁶ cells/well in 2 mL of DMEM. Cells were transduced with 1 μL, 5 μL, 10 μL of each lentiviral vector (Lv-CMV-GFP, Lv-HSV-tk-ts, Lv-HSV-tk-ts-opt, Lv-CMV-HSV-tk) in duplicate wells 24 hours later. 3 days post-transduction, one well from each transduction was harvested for DNA using the DNeasy Blood & Tissue Kit (Qiagen) following the manufacturer's instructions with an extended incubation time to 30 min at 56 ° C. Remaining cells were harvested 6 days post-transduction as previously described. 100 ng genomic DNA was amplified during psi qPCR quantification using 20 nM fwd/rev primers and SYBR Green PCR master mix (Applied Biosystems) alongside a standard curve based on serial dilutions of pSico plasmid DNA to determine psi copy number. 100 ng genomic DNA was amplified during albumin qPCR quantification using 100 nM fwd/rev primers and SYBR Green PCR master mix (Applied Biosystems) alongside a standard curve based on HEK 293T genomic DNA to determine the albumin copy number for estimation of cell number.

The following qPCR cycling protocol was used for both: initial ramp-up at 50° C. for 2 minutes and 95° C. for 20 seconds, 40 cycles of 95° C. for 3 seconds, and 60° C. for 30 seconds and finally a melting curve with temperatures cycling between 95° C. and 60° C. (95° C. for 15 seconds, 60° C. for 1min, 95° C. for 15 seconds, and 60° C. for 15 seconds). Infectivity titres were determined based on previously described methods (Kutner, R. H. et al (2009) Nature Protocols 4 495-505). Psi and albumin primers previously designed by Charrier et al. (2007) Gene Therapy 14 415-428.

Psi-Fwd
5′-CAGGACTCGGCTTGCTGAAG-3′,
Psi-Rev
5′-TCCCCCGCTTAATACTGACG-3′
Albumin-Fwd
5′-GCTGTCATCTCTTGTGGGCTGT-3,
Albumin-Rev
5′-ACTCATGGGAGCTGCTGGTTC-3′.

Lentiviral vectors pSico (negative control), 3′ER-HSVtk, 3′ER-HSVtk opt, or HSVtk (positive control) driven by the TBG promoter were titrated in H EK293T cells to determine infectious titres. Genomic DNA was isolated as above 3 days after transduction with 5 μl or 10 μl of lentiviral vector/well in triplicate wells and 20 ng genomic DNA was used as a template in qPCR reaction containing 2× TaqMan Fast Advanced Master Mix (Thermo Fisher Scientific) using 20 nM WPRE-Fwd and 20nM WPRE-Rev primers (WPRE-Fwd 5′-GGCACTGACAATTCCGTGGT-3′, WPRE-Rev 5′-AGGGACGTAGCAGAAGGACG-3′) and 100 nM WPRE probe 5′6FAM-ACGTCCTTTCCATGGCTGCTCGC-TAM3′. WPRE primer and probe sequences previously designed by Charrier et al. (2007) Gene Therapy 14 415-428. WPRE copy numbers were determined using a standard curve of serial dilutions of pSico plasmid DNA. qPCR cycling conditions were; 50° C. for 2 min, 95° C. for 20 sec, 40 cycles of 95° C. for 3 sec and 60° C. for 30 sec. Albumin primers were used in qPCR reactions using 100 ng genomic DNA as template to estimate cell numbers compared to a standard curve of serially diluted genomic DNA isolated from HEK 293T cells.

### Statistical Analysis

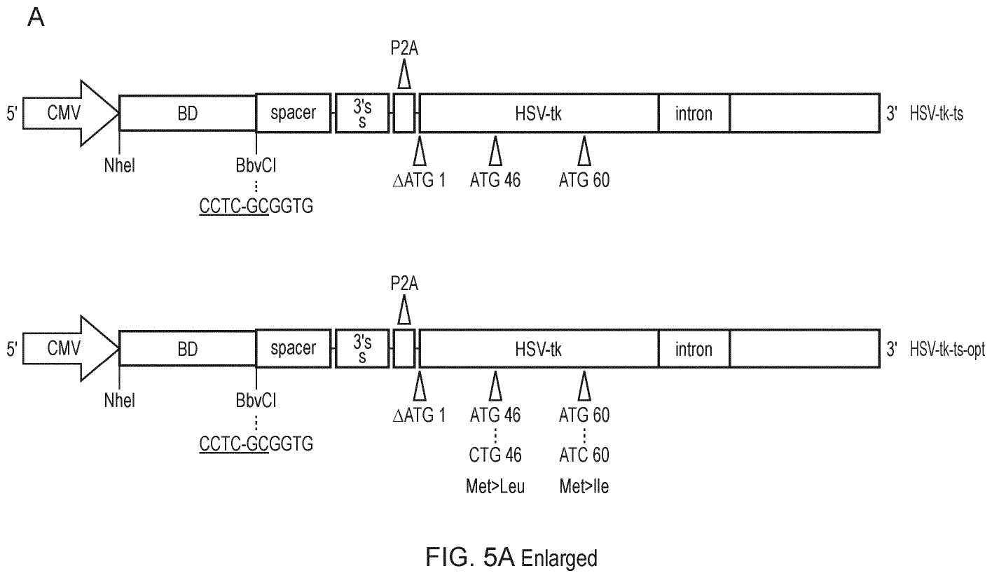

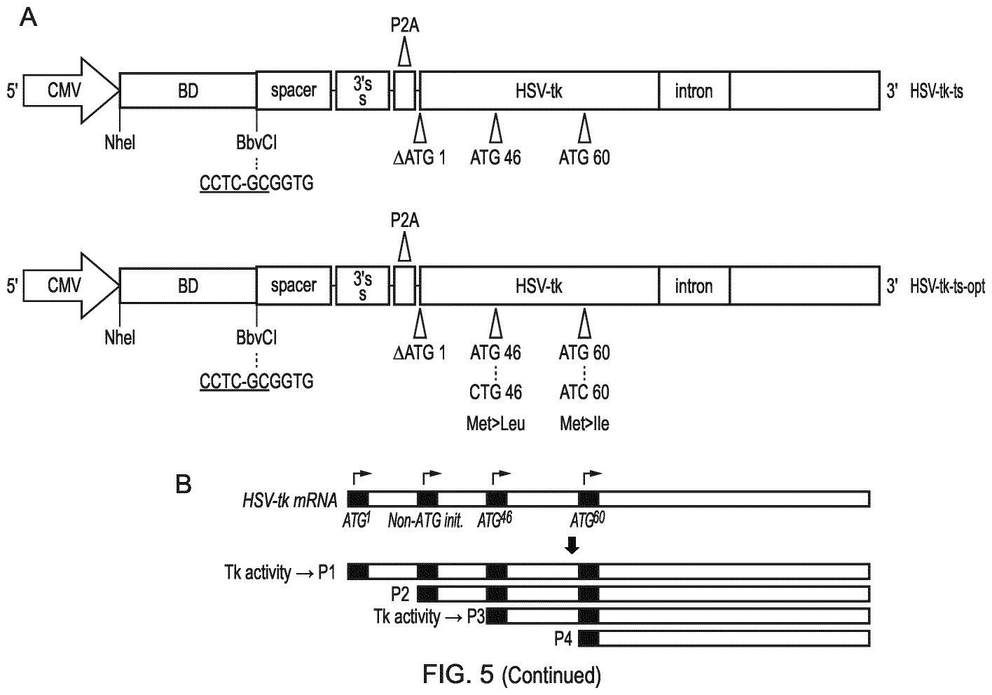

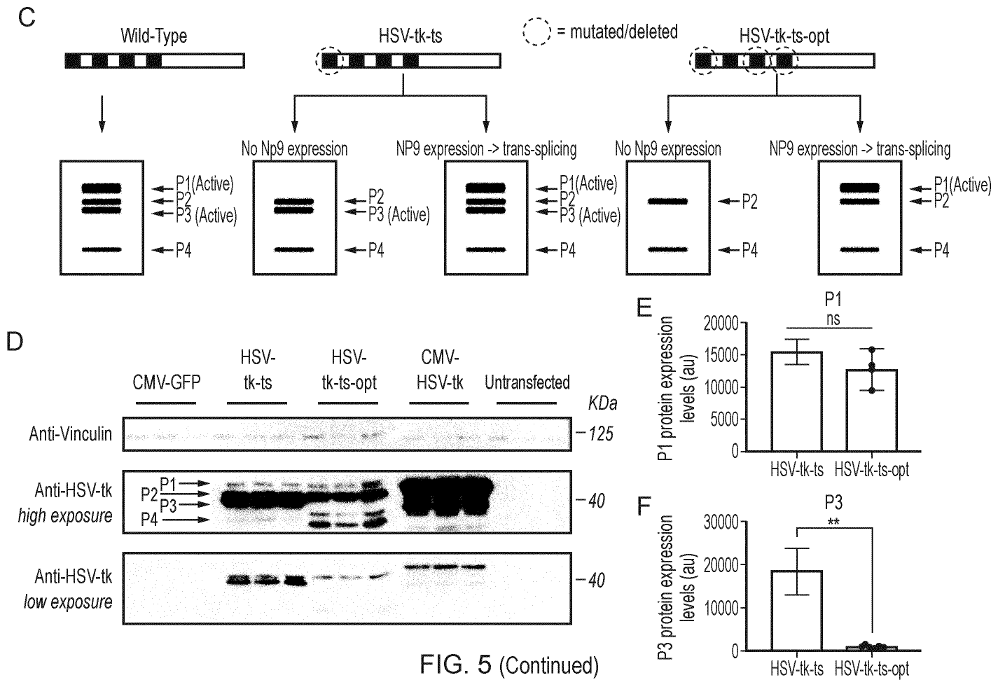

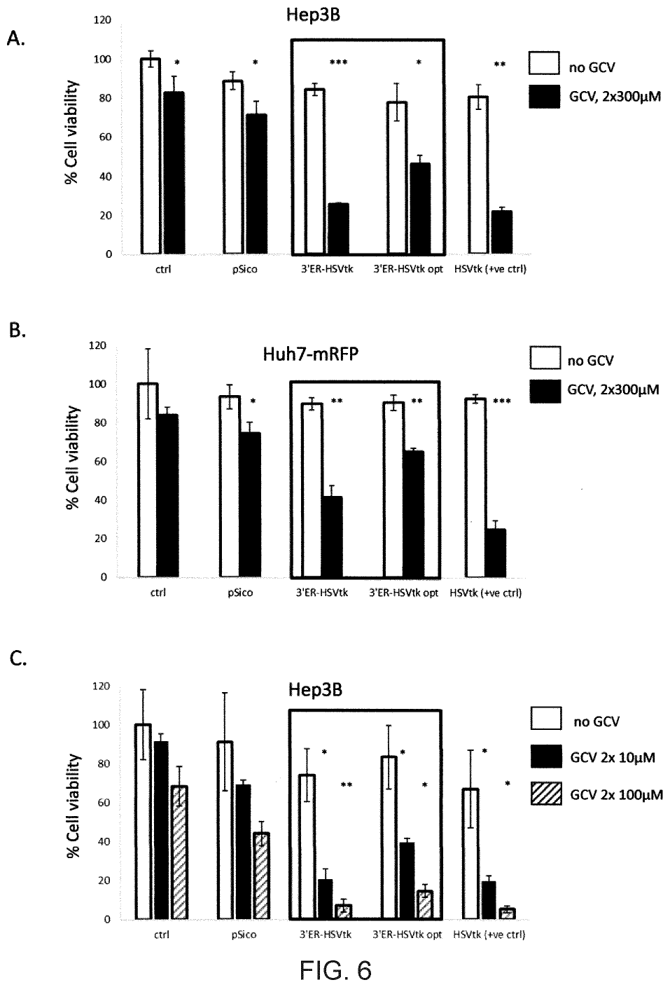

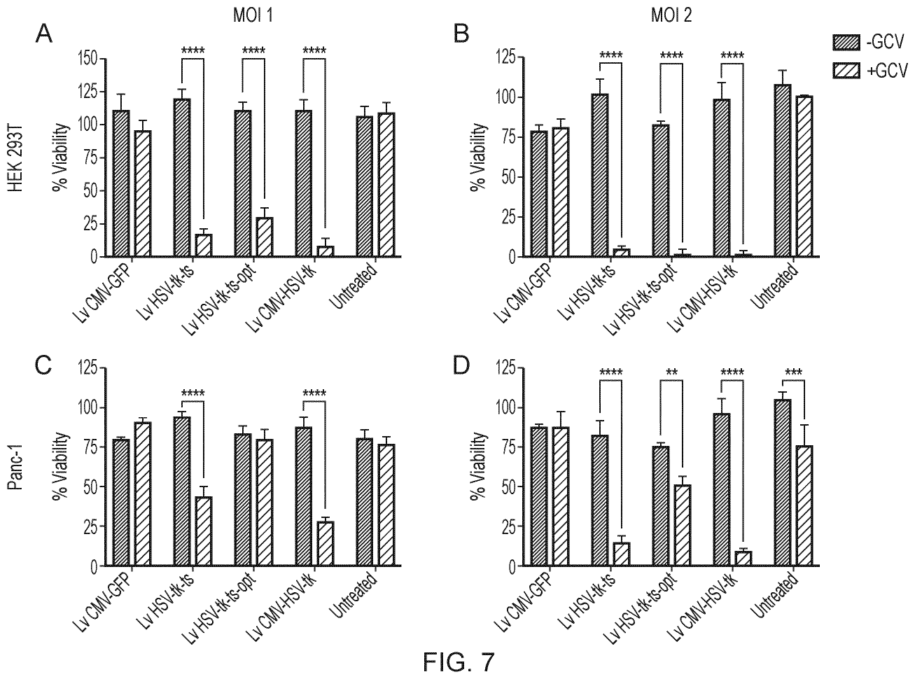

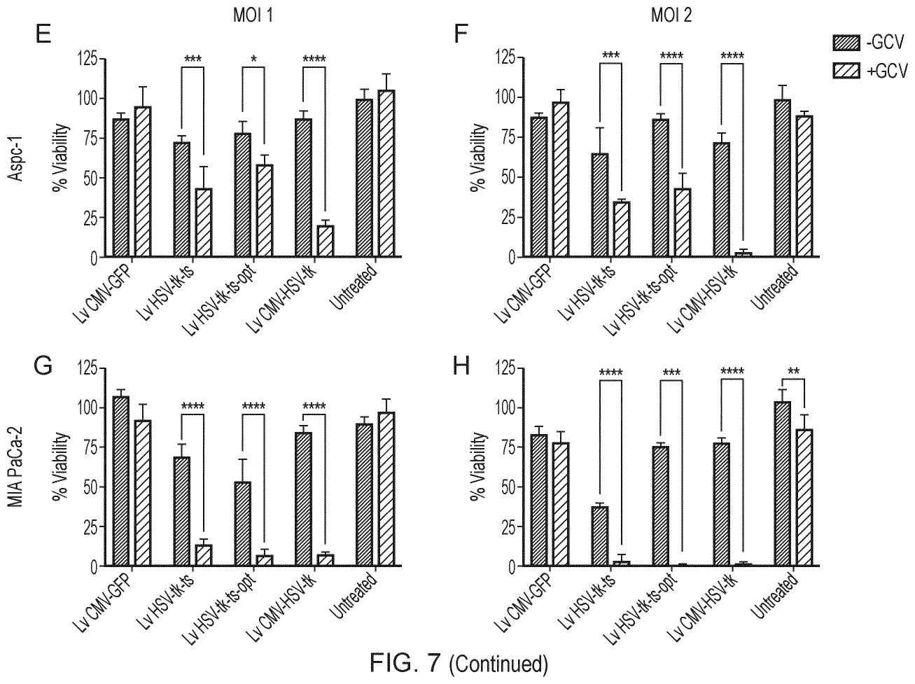

Statistical analyses in FIGS. 5 and 7 were performed using Prism 7 (Graphpad software). HSV-tk P1, P3 protein expression levels compared using a two-tailed unpaired t-test, assuming parametric distributions. In FIG. 7, all MTT cell viability data compared using a two-way ANOVA followed by Tukey's post-hoc multiple comparisons test. In FIG. 6, statistical analysis was performed using Student's t-Test, (Excel), assuming unequal variances. All p-values <0.05 were considered significant. All replicates used in statistical analysis are biological replicate wells.

### Results

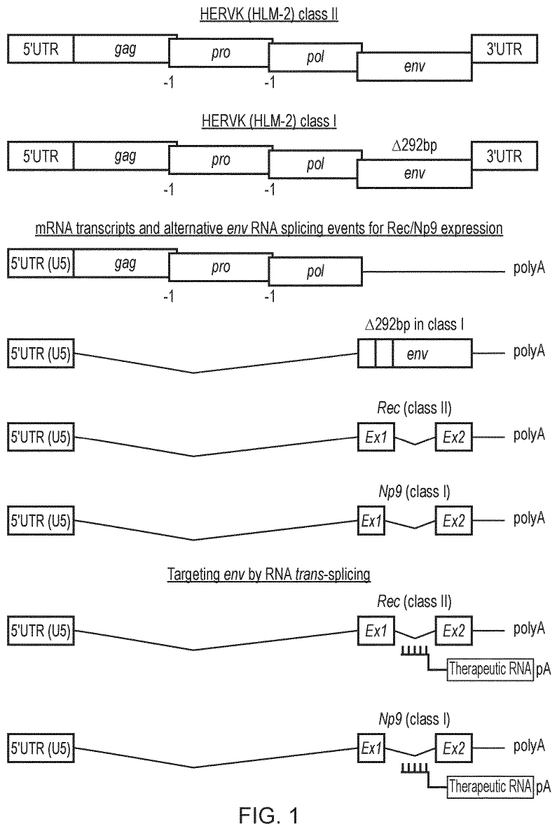

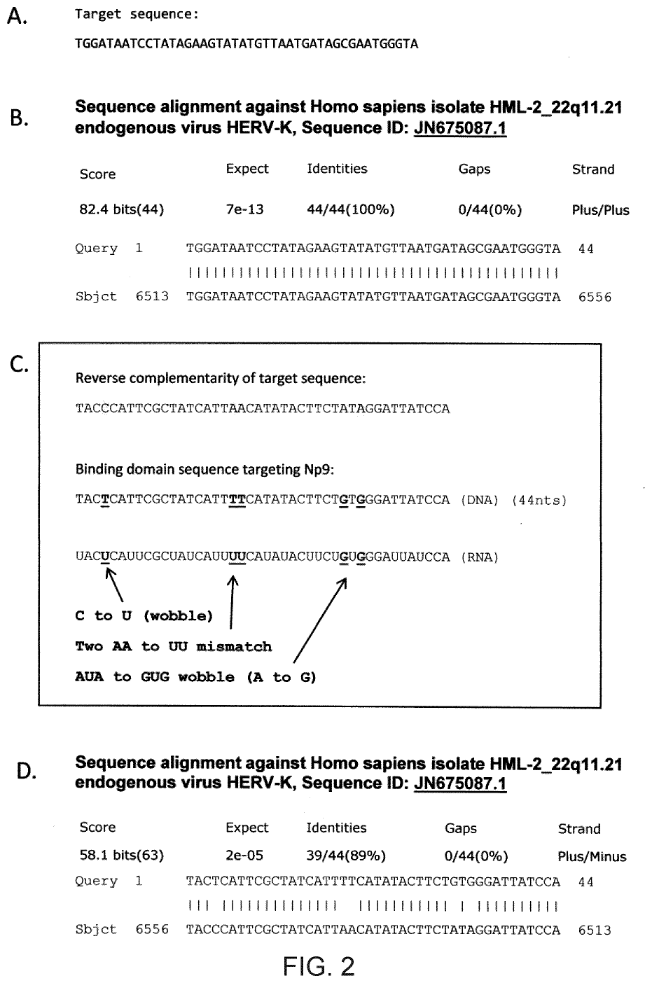

To target HCC or PDAC, we generated RNA trans-splicing constructs with a novel binding domain directed to HERV-K env pre-mRNA including an incomplete Herpes Simplex virus thymidine kinase gene (FIG. 1). Four wobble bases (C to U and A to G), or two mismatches (2×A to U) were included in the binding domain sequence to generate a favourable structure and avoid potential RNA editing (Chalupnikova et al (2013) Methods in Molecular Biology, 942, 291-314). Sequence alignment showed 89% complementarity to the HERV-K target sequence (Genbank Sequence ID: JN675087.1) (FIG. 2D).

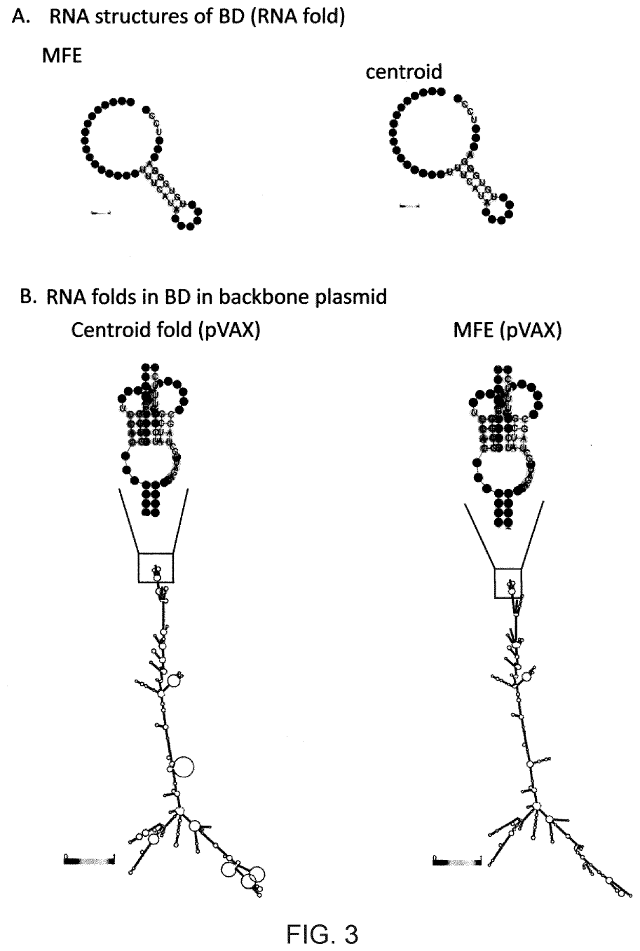

RNA secondary structure predictions of the isolated binding domain showed similar structures with a large number of unbound nucleotides for both minimum free energy (MFE) or centroid RNA secondary structure predictions when analysed by RNA fold (FIG. 3A). To confirm that the binding domain contained unstructured regions within the RNA trans-splicing cassette backbone, the RNA binding domain was refolded in the context of the RNA trans-splicing cassette. MFE and centroid folds confirmed stretches of up to 18 unbound nucleotides when folded within the RNA trans-splicing cassette (FIG. 3B).

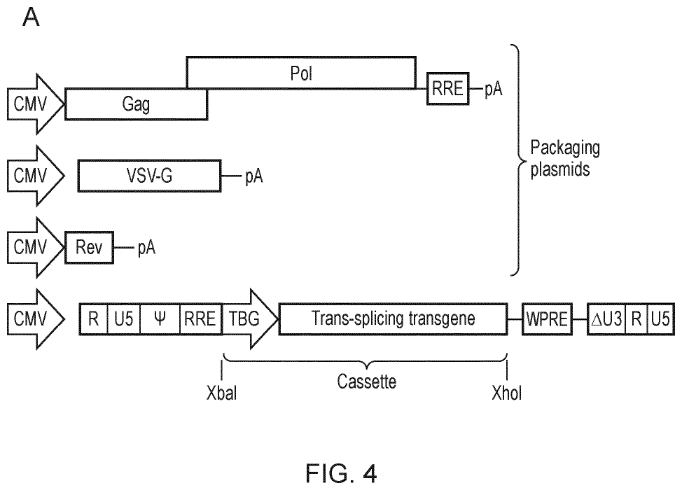

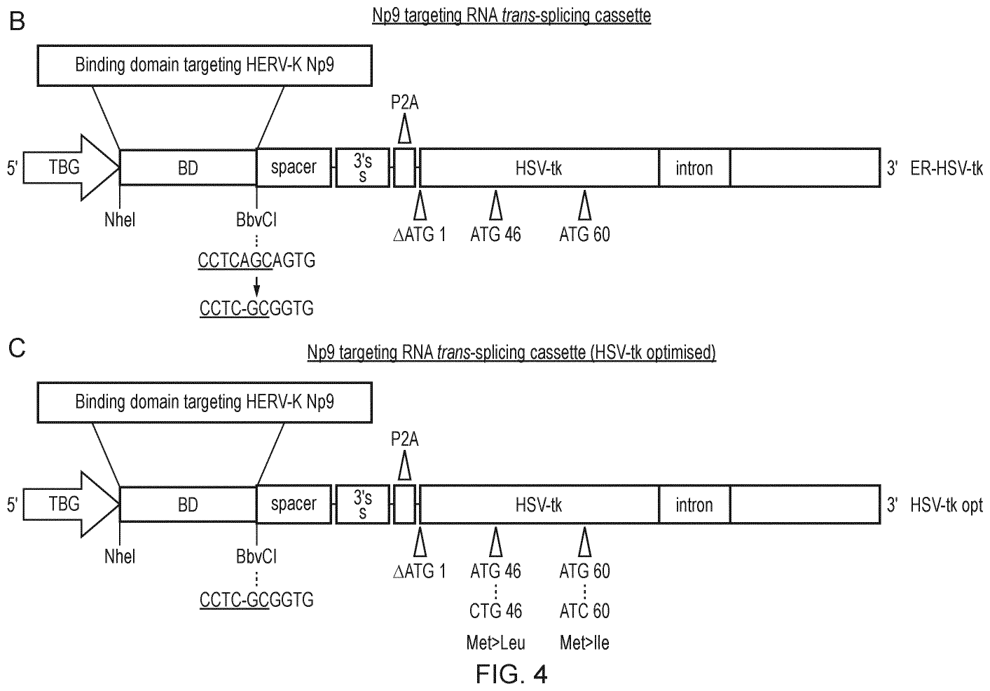

To further improve the RNA trans-splicing approach, Np9-targeting was combined with mutations within the RTM to reduce potential off-target splicing in cis (FIG. 4B top) and aberrant HSV-tk peptide production from the second (ATG 46) and third (ATG 60) canonical HSV-tk translational initiation codons. (FIG. 4B bottom).

To test the impact of the HSV-tk translational initiation mutants on HSV-tk isoform production, the Np9-targeting RNA trans-splicing construct HSV-tk-ts opt was tested in transfection studies in HEK293T cells compared to HSV-tk-ts harbouring wild type ATG 46 and ATG 60 but lacking the first ATG start codon of HSV-tk (FIG. 5A), alongside wild type HSV-tk containing all canonical HSV-tk translational initiation sites serving as a positive control, and a negative control construct expressing GFP from the CMV promoter. Translation products generated from the different HSV-tk translational initiation sites are shown in FIG. 5B and predicted HSV-tk isoforms generated from the different constructs are depicted in FIG. 5C. Western blot analysis revealed similar levels of P1 production when comparing HSV-tk-ts and HSV-tk-ts-opt (FIG. 5D), suggesting successful trans-splicing and subsequent HSV-tk P1 production driven from the Np9 translational initiation codon. Quantification of bands normalised to levels of vinculin (loading control) confirmed this observation (P<0.2877) (FIG. 5E). In contrast, expression levels of P3 were significantly reduced after transfection with HSV-tk-ts-opt compared to HSV-tk-ts where P3 isoform expression is overexpressed compared to wild-type HSV-tk (FIG. 5D). Quantification revealed an 18-fold loss of P3 expression in cells transfected with HSV-tk-ts-opt compared to HSV-tk-ts (P=0.0047) confirming a reduction in aberrant HSV-tk peptide production with our optimised RNA trans-splicing construct HSV-tk-ts-opt.

### Trans-Splicing Lentiviral Vectors Induce Cellular Death in Liver and Pancreatic Cancer Cell Lines

To test the effect on cell viability of Np9-targeting RNA trans-splicing lentiviral vectors, we evaluated the effect in MTT assay and compared vector transduced cells before and after GCV treatment for each vector type. In the hepatocellular carcinoma cell line Hep3B, a significant drop in cell viability was observed after transduction with the trans-splicing vector 3′ER-HSV-tk with 25.54±0.48% (P<0.001) viable cells after treatment with two doses of GCV at a concentration of 300 μM, comparable with the HSV-tk positive control vector with 21.67±2.21% (P<0.01) cells surviving after GCV treatment. With the tk-optimised trans-splicing vector, 3′ER-HSV-tk-opt, viability was significantly reduced to 46.01±4.39% (P<0.05) (FIG. 6A).

A similar trend was seen in HuH7-mRFP cells, where cell viability was reduced to 41.33±6.11% (P<0.01), or 64.96±1.77% (P<0.001) in cells transduced with trans-splicing vector 3′ER-HSV-tk or 3′ER-HSV-tk-opt after GCV treatment respectively. In HuH7-mRFP cells transduced with the HSV-tk positive control vector, 24.67±4.73% (P<0.001) of cells remained viable after addition of GCV (FIG. 6B).

In Hep3B cells, a significant drop in cell viability was observed when lowering the GCV concentration to two doses of 10 μM or 100 μM GCV, with remaining viable cells of 20.50±5.5% (P<0.05) for 3′ER-HSV-tk and 39.58±2.07% (P<0.01) 3′ER-HSV-tk-opt in combination with 10 μM GCV treatment. This was further reduced to 7.15±3.3% (P<0.01) and 14.62±3.38% (P<0.05) cell viability for 3′ER-HSV-tk and 3′ER-HSV-tk-opt respectively after two doses of 100 μM GCV. At these GCV concentrations, 19.55±2.90% (P<0.05) and 5.24±1.71% (P<0.05) of cells survived transduction with the HSV-tk positive control vector in combination with 10 μM or 100 μM respectively (FIG. 6C).

Next, we tested the Np9-targeting RNA trans-splicing lentiviral vectors in HEK 293T cells, which were transduced at an MOI of 1 and 2, treated with 1000 μM GCV, and subsequently analysed for cell viability by MTT assay. Following treatment with GCV, the trans-splicing lentiviral vectors, Lv HSV-tk-ts and Lv HSV-tk-ts-opt, at an MOI of 1 significantly reduced cell viability by 81.31±3.25% (P<0.0001) and 68.80±5.54% (P<0.0001) respectively, comparable to the 89.73±3.83% (P<0.0001) loss in cell viability in samples treated with GCV and the positive control CMV-HSV-tk (FIG. 7A). At an MOI of 2, the trans-splicing constructs induced a near-complete loss of cell viability with only 6.19±1.27% and 2.96±2.08% cells remaining in Lv HSV-tk-ts and Lv HSV-tk-ts-opt treated samples (FIG. 7B).

In addition, the efficacy of the trans-splicing lentiviral vectors was evaluated in pancreatic cancer cell lines. MIA Paca-2, Panc-1, and Aspc-1 cells were transduced with either Lv CMV-GFP, Lv HSV-tk-ts, Lv HSV-tk-ts-opt, Lv CMV-HSV-tk at an MOI of 1 or 2 and treated two days later with 1000 μM GCV. Both trans-splicing vectors resulted in significant losses in cell viability across all cell types tested at both MOls in the presence of GCV, except for Lv HSV-tk-ts-opt at an MOI of 1 in Panc-1 cells where no significant effect was seen (FIG. 7C, 7D, 7E, 7F, 7G, 7H).

In Panc-1 cells, Lv HSV-tk-ts and Lv HSV-tk-ts-opt showed HSV-tk/GCV-induced cytotoxicity starting at an MOI of 1 with 44.37±5.80% (P<0.0001) and 80.20±5.16% (P=0.8408) cell survival respectively, but showed highly significant cytotoxicity at an MOI of 2 with only 16.94±2.13% (P<0.0001) and 52.38±4.95% (P=0.0011) cell viability respectively (FIG. 7C, 7D). The trans-splicing lentiviral vectors (Lv HSV-tk-ts and Lv HSV-tk-ts-opt) were also efficacious in Aspc-1 cells, to an even greater degree than in Panc-1 cells, with a survival of only 45.16±11.99% (P=0.0005) and 59.69±5.21% (P=0.0244) of cells when treated at an MOI of 1 and only 35.67±1.21% (P=0.0002) and 43.95±8.47% (P<0.0001) of cells when treated at an MOI of 2 (FIG. 7E, 7F). Finally, in MIA PaCa-2 cells, as in HEK 293T cells, there is nearly a complete loss of all cell viability after treatment with the trans-splicing lentiviral vectors and 1000 μM GCV. At an MOI of 1, only 14.07±2.82% (P<0.0001) and 8.03±2.19% (P<0.0001) of cells remain after treatment with Lv HSV-tk-ts and Lv HSV-tk-ts-opt respectively (FIG. 7G). At an MOI of 2 this effect is even more prominent as only 4.11±4.54% (P<0.0001) and 1.22±0.31% (P=0.0002) of cells remain viable after treatment with Lv HSV-tk-ts and Lv HSV-tk-ts-opt respectively, comparable to those observed in samples treated with the positive control Lv CMV-HSV-tk where only 2.09±0.53% (P<0.0001) cell viability was observed after treatment (FIG. 7H).

Taken together, this confirms the efficacy of targeting HERV-K Env by RNA trans-splicing to kill liver and pancreatic cancer cells.

```
Reference Sequences
TGGATAATCCTATAGAAGTATATGTTAATGATAGCGAATGGGTA
SEQ ID NO: 1-HERV-K target sequence of binding region (DNA)
UGGAUAAUCCUAUAGAAGUAUAUGUUAAUGAUAGCGAAUGGGUA
SEQ ID NO: 2-HERV-K target sequence of binding region (RNA)
TACCCATTCGCTATCATTAACATATACTTCTATAGGATTATCCA
SEQ ID NO: 3-Reverse complement of HERV-K target sequence (DNA)
UACCCAUUCGCUAUCAUUAACAUAUACUUCUAUAGGAUUAUCCA
SEQ ID NO: 4-Reverse complement of HERV-K target sequence (RNA)
UACUCAUUCGCUAUCAUUUUCAUAUACUUCUGUGGGAUUAUCCA
SEQ ID NO: 5-Modified reverse complement of HERV-K target sequence (RNA)
001 masypchqha safdqaarsr ghsnrrtalr prrqqeatev rpeqkmptll rvyidgphgm
061 gkttttqllv algsrddivy vpepmtywrv lgasetiani yttqhrldqg eisagdaavv
121 mtsagitmgm pyavtdavla phiggeagss happpaltli fdrhpiaall cypaarylmg
181 smtpqavlaf valipptlpg tnivlgalpe drhidrlakr qrpgerldla mlaairrvyg
241 llantvrylq gggswredwg qlsgtavppq gaepqsnagp rphigdtlft lfrapellap
301 ngdlynvfaw aldvlakrlr pmhvfildyd qspagcrdal lqltsgmvqt hvttpgsipt
361 icdlartfar emgean
SEQ ID NO: 6-HSV Thymidine Kinase (HSV-tk) amino acid sequence (AAC16235.1)
  306 atggc ttcgtacccc tgccatcaac acgcgtctgc gttcgaccag gctgcgcgtt
  361 ctcgcggcca tagcaaccga cgtacggcgt tgcgccctcg ccggcagcaa gaagccacgg
  421 aagtccgccc ggagcagaaa atgcccacgc tactgcgggt ttatatagac ggtccccacg
  481 ggatggggaa aaccaccacc acgcaactgc tggtggccct gggttcgcgc gacgatatcg
  541 tctacgtacc cgagccgatg acttactggc gggtgctggg ggcttccgag acaatcgcga
  601 acatctacac cacacaacac cgcctcgacc agggtgagat atcggccggg gacgcggcgg
  661 tggtaatgac aagcgcccag ataacaatgg gcatgcctta tgccgtgacc gacgccgttc
  721 tggctcctca tatcgggggg gaggctggga gctcacatgc cccgcccccg gccctcaccc
  781 tcatcttcga ccgccatccc atcgccgccc tectgtgcta cccggccgcg cgatacctta
  841 tgggcagcat gaccccccag gccgtgctgg cgttcgtggc cctcatcccg ccgaccttgc
  901 ccggcacaaa catcgtgttg ggggcccttc cggaggacag acacatcgac cgcctggcca
  961 aacgccagcg ccccggcgag cggcttgacc tggctatgct ggccgcgatt cgccgcgttt
1021 acgggctgct tgccaatacg gtgcggtatc tgcagggcgg cgggtcgtgg cgggaggatt
1081 ggggacagct ttcggggacg gccgtgccgc cccagggtgc cgagccccag agcaacgcgg
1141 gcccacgacc ccatatcggg gacacgttat ttaccctgtt tegggccccc gagttgctgg
1201 cccccaacgg cgacctgtat aacgtgtttg cctgggcctt ggacgtcttg gccaaacgcc
1261 tccgtcccat gcacgtcttt atcctggatt acgaccaatc gcccgccggc tgccgggacg
1321 ccctgctgca acttacctcc gggatggtcc agacccacgt caccacccca ggctccatac
1381 cgacgatctg cgacctggeg cgcacgtttg cccgggagat gggggaggct aactga
SEQ ID NO: 7-HSV Thymidine Kinase (HSV-tk) coding sequence (AF057310.1)
ASYPCHQHASAFDQAARSRGHSNRRTALRPRRQQEATEVRPEQKLPTLLRVYIDGPHGI
GKTTTTQLLVALGSRDDIVYVPEPMTYWRVLGASETIANIYTTQHRLDQGEISAGDAAVV
MTSAQITMGMPYAVTDAVLAPHIGGEAGSSHAPPPALTLIFDRHPIAALLCYPAARYLMG
SMTPQAVLAFVALIPPTLPGTNIVLGALPEDRHIDRLAKRORPGERLDLAMLAAIRRVYG
LLANTVRYLQGGGSWREDWGQLSGTAVPPQGAEPQSNAGPRPHIGDTLFTLFRAPELLAP
NGDLYNVFAWALDVLAKRLRPMHVFILDYDQSPAGCRDALLOLTSGMVQTHVTTPGSIPT
ICDLARTFAREMGEAN
SEQ ID NO: 8-modified HSV Thymidine Kinase (HSV-tk) amino acid sequence
  309 gc ttcgtacccc tgccatcaac acgcgtctgc gttcgaccag gctgcgcgtt
  361 ctcgcggcca tagcaaccga cgtacggcgt tgcgccctcg ccggcagcaa gaagccacgg
  421 aagtccgccc ggagcagaaa ctgcccacgc tactgcgggt ttatatagac ggtccccacg
  481 ggatcgggaa aaccaccacc acgcaactgc tggtggccct gggttcgcgc gacgatatcg
  541 tctacgtacc cgagccgatg acttactggc gggtgctggg ggcttccgag acaatcgcga
  601 acatctacac cacacaacac cgcctcgacc agggtgagat atcggccggg gacgcggcgg
  661 tggtaatgac aagcgcccag ataacaatgg gcatgcctta tgccgtgacc gacgccgttc
  721 tggctcctca tategggggg gaggctggga gctcacatgc cccgcccccg gccctcaccc
  781 tcatcttcga ccgccatccc atcgccgccc tectgtgcta cccggccgcg cgatacctta
  841 tgggcagcat gaccccccag gccgtgctgg cgttcgtggc cctcatcccg ccgaccttgc
  901 ccggcacaaa catcgtgttg ggggcccttc cggaggacag acacatcgac cgcctggcca
  961 aacgccagcg ccccggcgag cggcttgacc tggctatgct ggccgcgatt cgccgcgttt
1021 acgggctgct tgccaatacg gtgcggtatc tgcagggcgg cgggtcgtgg cgggaggatt
1081 ggggacagct ttcggggacg gccgtgccgc cccagggtgc cgagccccag agcaacgcgg
1141 gcccacgacc ccatatcggg gacacgttat ttaccctgtt tcgggccccc gagttgctgg
1201 cccccaacgg cgacctgtat aacgtgtttg cctgggcctt ggacgtcttg gccaaacgcc
1261 tccgtcccat gcacgtcttt atcctggatt acgaccaatc gcccgccggc tgccgggacg
1321 ccctgctgca acttacctcc gggatggtcc agacccacgt caccacccca ggctccatac
1381 cgacgatctg cgacctggeg cgcacgtttg cccgggagat gggggaggct aactga
SEQ ID NO: 9-modified HSV Thymidine Kinase (HSV-tk) coding sequence
GGTGACGAAAA CGTGCTATCA GTTCGCTCCC CCACTCCC
SEQ ID NO: 10-Spacer sequence
GSGATNFSLL KQAGDVEENP GP
SEQ ID NO: 11-P2A cleavage sequence
GGGCCTGGGC CTGGGTACTA ACACGATCGT TTTTTTCCCT TTTTTTCCAG G
SEQ ID NO: 12-Trans-splicing domain sequence
```

| | |
|---|---|
| GCUAGCUACU CAUUCGCUAU CAUUUUCAUA UACUUCUGUG GGAUUAUCCA CCUCGCGGUG | 60 |
| ACGAAAACGU GCUAUCAGUU CGCUCCCCCA CUCCCGCUUU CAUUUUUGUC UUGUCUUUUU | 120 |
| UUAACCUGGG CCUGGGCCUG GGUACUAACA CGAUCGUUUU UUUCCCUUUU UUUCCAGGGG | 180 |
| AAGCGGAGCU ACUAACUUCA GCCUGCUGAA GCAGGCUGGA GACGUGGAGG AGAACCCUGG | 240 |
| GCCUGCUUCG UACCCCUGCC AUCAACACGC GUCUGCGUUC GACCAGGCGG CGCGAUCACG | 300 |
| GGGACACAGC AACCGACGGA CGGCGUUGCG CCCUCGCCGG CAGCAAGAAG CCACGGAAGU | 360 |
| CCGCCCGGAG CAGAAAAUGC CCACGCUACU GCGGGUUUAU AUAGACGGUC CCCACGGGAU | 420 |
| GGGGAAAACC ACCACCACGC AACUGCUGGU GGCCCUGGGU UCGCGCGACG AUAUCGUCUA | 480 |
| CGUACCCGAG CCGAUGACUU ACUGGCGGGU GCUGGGGGCU UCCGAGACAA UCGCGAACAU | 540 |
| CUACACCACA CAACACCGCC UCGACCAGGU AAGUAUCAAG GUUACAAGAC AGGUUUAAGG | 600 |
| AGACCAAUAG AAACUGGGCU UGUCGAGACA GAGACGACUC UUGCGUUUCU GAUAGGCACC | 660 |
| UAUUGGUCUU ACUGACAUCC ACUUUGCCUU UCUCUCCACA GGGUGAGAUA UCGGCCGGGG | 720 |
| ACGCGGCGGU GGUAAUGACA AGCGCCCAGA UAACAAUGGG CAUGCCUUAU GCCGUGACCG | 780 |
| ACGCCGUUCU GGCUCCUCAU AUCGGGGGGG AGGCUGGGAG CUCACAUGCC CCUCCUCCGG | 840 |
| CCCUCACCCU CAUCUUCGAC CGCCAUCCCA UCGCCGCCCU CCUGUGCUAC CCGGCCGCGC | 900 |
| GAUACCUUAU GGGCAGCAUG ACCCCCCAGG CCGUGCUGGC GUUCGUGGCC CUCAUCCCGC | 960 |
| CGACCUUGCC CGGCACAAAC AUCGUGUUGG GGGCCCUUCC GGAGGACAGA CACAUCGACC | 1020 |
| GCCUGGCCAA ACGCCAGCGC CCCGGCGAGC GGCUUGACCU GGCUAUGCUG GCCGCGAUUC | 1080 |
| GCCGCGUUUA CGGGCUGCUU GCCAAUACGG UGCGGUAUCU GCAGGGCGGC GGGUCGUGGC | 1140 |
| GGGAGGAUUG GGGACAGCUU UCGGGGACGG CCGUGCCGCC UCAGGGUGCC GAGCCUCAGA | 1200 |
| GCAACGCGGG CCCACGACCC CAUAUCGGGG ACACGUUAUU UACCCUGUUU CGGGCCCCCG | 1260 |
| AGUUGCUGGC CCCCAACGGC GACCUGUAUA ACGUGUUUGC CUGGGCCUUG GACGUCUUGG | 1320 |
| CCAAACGCCU CCGUCCCAUG CACGUCUUUA UCCUGGAUUA CGACCAAUCG CCCGCCGGCU | 1380 |
| GCCGGGACGC CCUGCUGCAA CUUACCUCCG GGAUGGUCCA GACCCACGUC ACCACCCCAG | 1440 |
| GCUCCAUACC GACGAUCUGC GACCUGGCGC GCACGUUUGC GCGGGAGAUG GGGGAGGCGA | 1500 |
| ACUGACUCGA G | 1511 |

SEQ ID NO: 13-RTM sequence

| | |
|---|---|
| 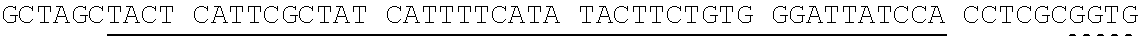 | 60 |
| 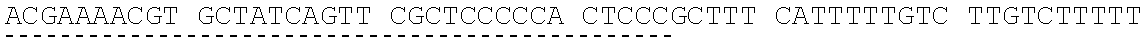 | 120 |
| 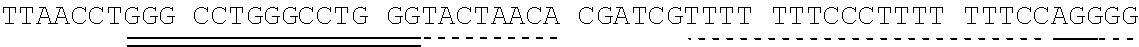 | 180 |
| 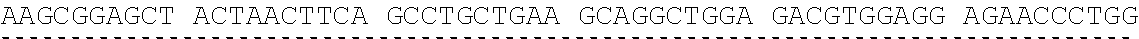 | 240 |
| 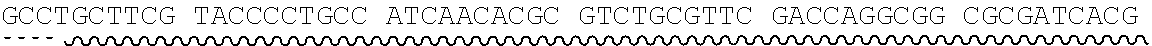 | 300 |
| 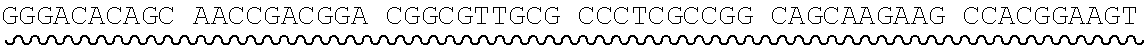 | 360 |
| 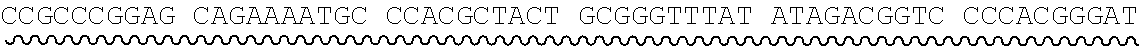 | 420 |
| 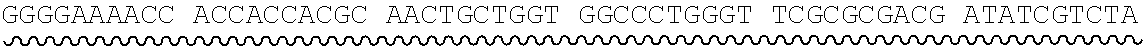 | 480 |
| 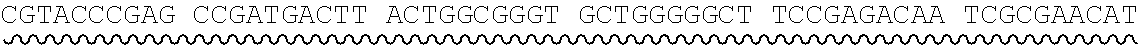 | 540 |
| 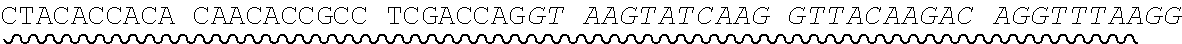 | 600 |
| 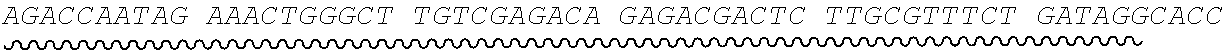 | 660 |
| 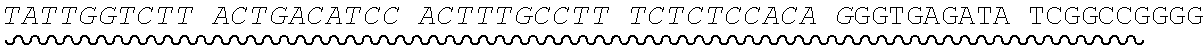 | 720 |
| 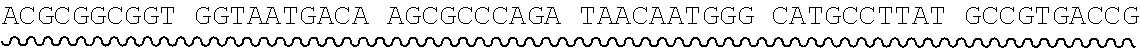 | 780 |
| 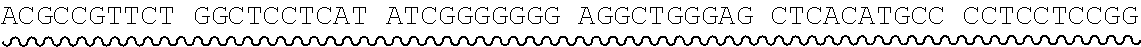 | 840 |
| 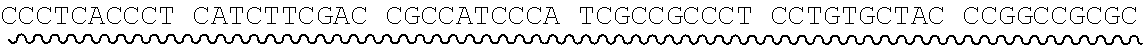 | 900 |
| 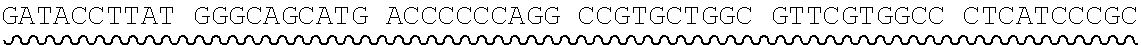 | 960 |
| 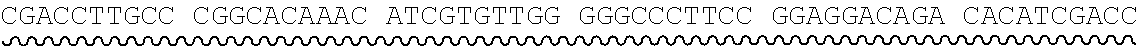 | 1020 |
| 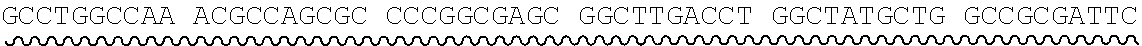 | 1080 |
| 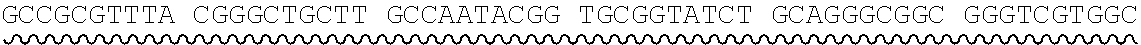 | 1140 |
| 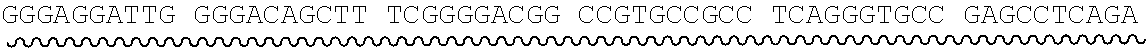 | 1200 |
| 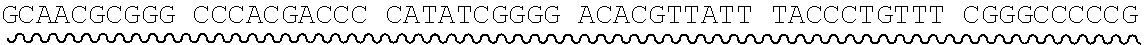 | 1260 |
| 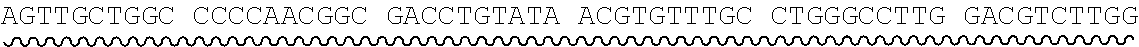 | 1320 |
| 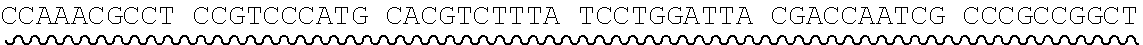 | 1380 |
| 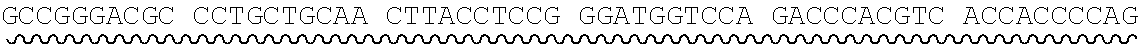 | 1440 |
| 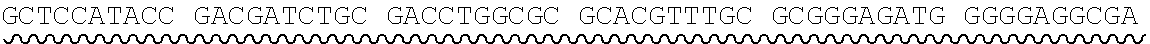 | 1500 |
| 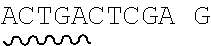 | 1511 |

| | | |
|---|---|---|
| Bases 1 to 6 | Nhe1 site | |
| Bases 7 to 50 | Binding domain | Solid underline |
| Bases 51 to 56 | Modified BbvC1 site | |
| Bases 57 to 95 | Spacer | Dotted underline |
| Bases 128 to 142 | ISE | Double underline |
| Bases 143 to 150 | Branch point | Dashed underline |
| Bases 150 to 156 | Pvu1 site | |
| Bases 157 to 175 | Polypyrimidine tract | Dotted + dashed underline |
| Bases 176 to 178 | Splice site | Solid underline |
| Bases 179 to 244 | P2A sequence | Dotted underline |
| Bases 245 to 1505 | HSVtk sequence | Wavy underline |
| Bases 569 to 711 | Intron | Italicised |
| Bases 1506 to 1511 | Xho1 site | |

SEQ ID NO: 14-Coding DNA sequence for RTM (SEQ ID NO: 13)

| | |
|---|---|
| GCUAGCUACU CAUUCGCUAU CAUUUUCAUA UACUUCUGUG GGAUUAUCCA CCUCGCGGUG | 60 |
| ACGAAAACGU GCUAUCAGUU CGCUCCCCCA CUCCCGCUUU CAUUUUUGUC UUGUCUUUUU | 120 |
| UUAACCUGGG CCUGGGCCUG GGUACUAACA CGAUCGUUUU UUUCCCUUUU UUUCCAGGGG | 180 |
| AAGCGGAGCU ACUAACUUCA GCCUGCUGAA GCAGGCUGGA GACGUGGAGG AGAACCCUGG | 240 |
| GCCUGCUUCG UACCCCUGCC AUCAACACGC GUCUGCGUUC GACCAGGCGG CGCGAUCACG | 300 |
| GGGACACAGC AACCGACGGA CGGCGUUGCG CCCUCGCCGG CAGCAAGAAG CCACGGAAGU | 360 |
| CCGCCCGGAG CAGAAACUGC CCACGCUACU GCGGGUUUAU AUAGACGGUC CCCACGGGAU | 420 |
| CGGGAAAACC ACCACCACGC AACUGCUGGU GGCCCUGGGU UCGCGCGACG AUAUCGUCUA | 480 |
| CGUACCCGAG CCGAUGACUU ACUGGCGGGU GCUGGGGGCU UCCGAGACAA UCGCGAACAU | 540 |
| CUACACCACA CAACACCGCC UCGACCAGGU AAGUAUCAAG GUUACAAGAC AGGUUUAAGG | 600 |
| AGACCAAUAG AAACUGGGCU UGUCGAGACA GAGACGACUC UUGCGUUUCU GAUAGGCACC | 660 |
| UAUUGGUCUU ACUGACAUCC ACUUUGCCUU UCUCUCCACA GGGUGAGAUA UCGGCCGGGG | 720 |
| ACGCGGCGGU GGUAAUGACA AGCGCCCAGA UAACAAUGGG CAUGCCUUAU GCCGUGACCG | 780 |
| ACGCCGUUCU GGCUCCUCAU AUCGGGGGGG AGGCUGGGAG CUCACAUGCC CCUCCUCCGG | 840 |
| CCCUCACCCU CAUCUUCGAC CGCCAUCCCA UCGCCGCCCU CCUGUGCUAC CCGGCCGCGC | 900 |
| GAUACCUUAU GGGCAGCAUG ACCCCCCAGG CCGUGCUGGC GUUCGUGGCC CUCAUCCCGC | 960 |
| CGACCUUGCC CGGCACAAAC AUCGUGUUGG GGGCCCUUCC GGAGGACAGA CACAUCGACC | 1020 |
| GCCUGGCCAA ACGCCAGCGC CCCGGCGAGC GGCUUGACCU GGCUAUGCUG GCCGCGAUUC | 1080 |
| GCCGCGUUUA CGGGCUGCUU GCCAAUACGG UGCGGUAUCU GCAGGGCGGC GGGUCGUGGC | 1140 |
| GGGAGGAUUG GGGACAGCUU UCGGGGACGG CCGUGCCGCC UCAGGGUGCC GAGCCUCAGA | 1200 |
| GCAACGCGGG CCCACGACCC CAUAUCGGGG ACACGUUAUU UACCCUGUUU CGGGCCCCCG | 1260 |
| AGUUGCUGGC CCCCAACGGC GACCUGUAUA ACGUGUUUGC CUGGGCCUUG GACGUCUUGG | 1320 |
| CCAAACGCCU CCGUCCCAUG CACGUCUUUA UCCUGGAUUA CGACCAAUCG CCCGCCGGCU | 1380 |
| GCCGGGACGC CCUGCUGCAA CUUACCUCCG GGAUGGUCCA GACCCACGUC ACCACCCCAG | 1440 |
| GCUCCAUACC GACGAUCUGC GACCUGGCGC GCACGUUUGC GCGGGAGAUG GGGGAGGCGA | 1500 |
| ACUGACUCGA G | 1511 |

SEQ ID NO: 15-RTM sequence with modified HSV-tk coding sequence

| | |
|---|---|
| 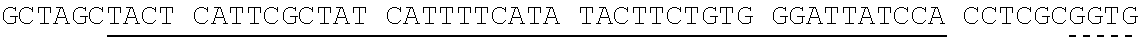 | 60 |
| 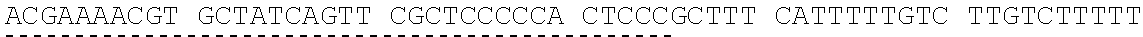 | 120 |
| 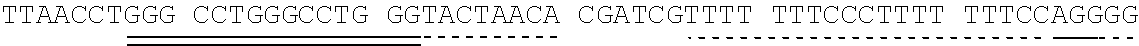 | 180 |
| 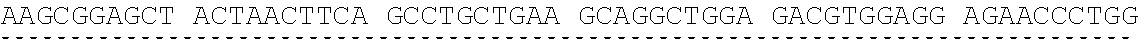 | 240 |
| 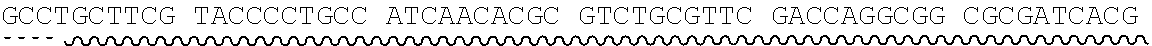 | 300 |
| 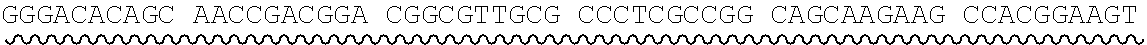 | 360 |
| 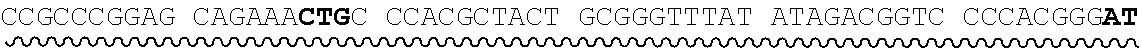 | 420 |
| 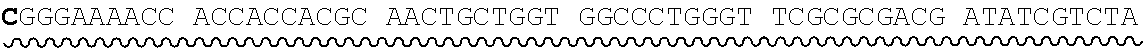 | 480 |
| 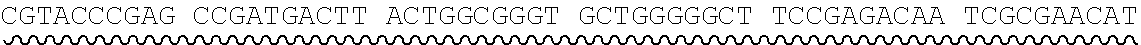 | 540 |
| 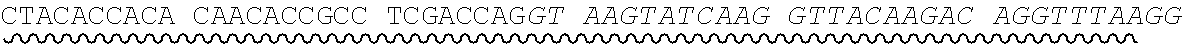 | 600 |
| 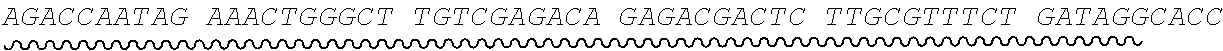 | 660 |
| 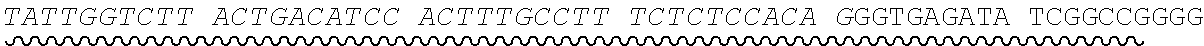 | 720 |
| 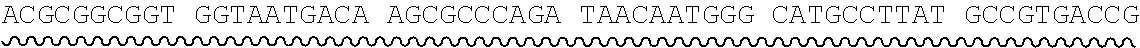 | 780 |
| 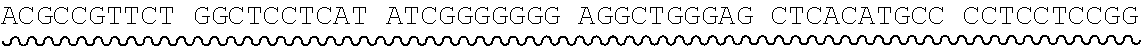 | 840 |
| 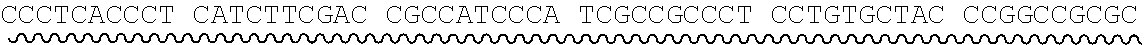 | 900 |
| 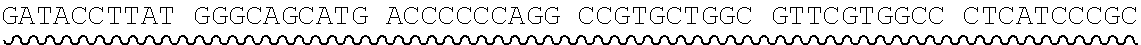 | 960 |
| 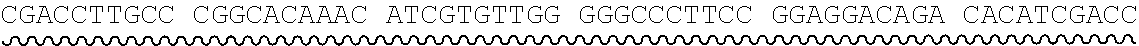 | 1020 |
| 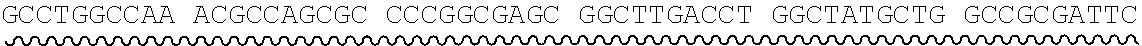 | 1080 |
| 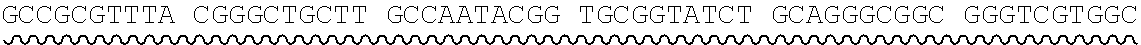 | 1140 |
| 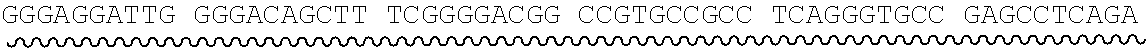 | 1200 |
| 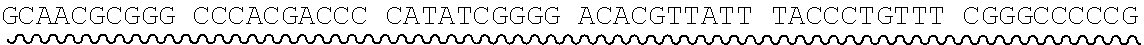 | 1260 |
| 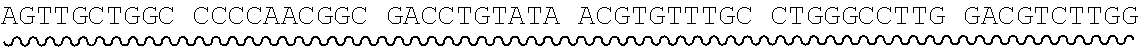 | 1320 |
| 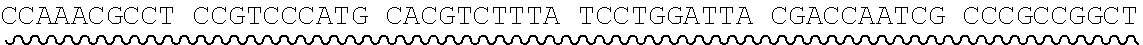 | 1380 |
| 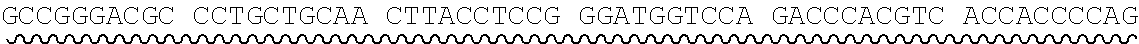 | 1440 |
| 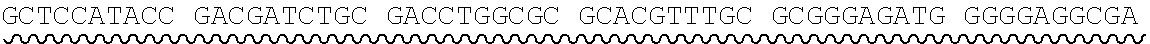 | 1500 |
| 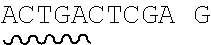 | 1511 |

| | | |
|---|---|---|
| Bases 1 to 6 | Nhe1 site | |
| Bases 7 to 50 | Binding domain | Solid underline |
| Bases 51 to 56 | Modified BbvC1 site | |
| Bases 57 to 95 | Spacer | Dotted underline |
| Bases 128 to 142 | ISE | Double underline |
| Bases 143 to 150 | Branch point | Dashed underline |
| Bases 150 to 156 | Pvu1 site | |
| Bases 157 to 175 | Polypyrimidine tract | Dotted + dashed underline |
| Bases 176 to 178 | Splice site | Solid underline |
| Bases 179 to 244 | P2A sequence | Dotted underline |
| Bases 245 to 1505 | HSVtk sequence | Wavy underline |
| Bases 377 to 380 | Mutated ATG | Bold |
| Based 419 to 421 | Mutated ATG | Bold |
| Bases 569 to 711 | Intron | Italicised |
| Bases 1506 to 1511 | Xho1 site | |

SEQ ID NO: 16-Coding DNA sequence for RTM with modified HSV-tk coding sequence (SEQ ID NO: 15)
GGAAGCGGAGCT ACTAACTTCA GCCTGCTGAA GCAGGCTGGA GACGTGGAGG AGAACCCTGG GCCT
SEQ ID NO: 17-nucleotide sequence encoding P2A peptide.

1. An RNA trans-splicing molecule (RTM) comprising;
(i) a binding region specific for a HERV pre-mRNA,
(ii) a trans-splicing domain and
(iii) a coding sequence for a suicide protein.

2. An RTM according to claim 1 wherein the binding region specifically binds to HERV pre-mRNA in a cell, such that the coding sequence is trans-spliced with the HERV mRNA, causing the suicide protein to be expressed in the cell.

3. An RTM according to claim 1 or claim 2 wherein the HERV pre-mRNA is a HERV-K pre-mRNA.

4. An RTM according to claim 3 wherein the HERV-K pre-mRNA is a HERV-K Np9 or HERV-K Rec pre-mRNA.

5. An RTM according to claim 3 wherein the binding region specifically binds to the nucleotide sequence of SEQ ID NO: 2 within the HERV-K Np9 or HERV-K Rec pre-mRNA.

6. An RTM according to any one of the preceding claims wherein the binding region comprises the nucleotide sequence of SEQ ID NO: 4 or a variant thereof.

7. An RTM according to claim 6 wherein the binding region comprises a nucleotide sequence that is a variant of SEQ ID NO: 4 with modifications relative to SEQ ID NO: 4 at one or more positions corresponding to positions 4, 19, 20, 32 and 34 of SEQ ID NO: 4.

8. An RTM according to claim 7 wherein the binding region comprises a nucleotide sequence that is a variant of SEQ ID NO: 4 with a C to U substitution at a position corresponding to position 4 of SEQ ID NO: 4; an A to U substitution at position corresponding to position 19; an A to U substitution at position corresponding to position 20; an A to G substitution at position corresponding to position 32; and an A to G substitution at position corresponding to position 34.

9. An RTM according to any one of the preceding claims wherein the binding region comprises the nucleotide sequence of SEQ ID NO: 5 or a variant thereof.

10. An RTM according to any one of the preceding claims wherein the suicide protein is Herpes simplex virus (HSV) thymidine kinase.

11. An RTM according to claim 10 wherein the HSV thymidine kinase comprises the amino acid sequence of SEQ ID NO: 6 or a variant thereof.

12. An RTM according to claim 10 or 11 wherein the HSV thymidine kinase comprises an amino acid sequence that is a variant of SEQ ID NO: 6 with modifications relative to SEQ ID NO: 6 at positions corresponding to positions 46 and 60 of SEQ ID NO: 6, optionally positions corresponding to positions 1, 46 and 60 of SEQ ID NO: 6.

13. An RTM according to claim 12 wherein the HSV thymidine kinase comprises an amino acid sequence that is variant of SEQ ID NO: 6 with an M to L substitution at a position corresponding to position 46; and an M to I substitution at a position corresponding to position 60 and optionally a deletion of M at a position corresponding to position 1 of SEQ ID NO: 6.

14. An RTM according to any of claims 10 to 13 wherein the HSV thymidine kinase comprises the amino acid sequence of SEQ ID NO: 8 or a variant thereof.

15. An RTM according to any one of the preceding claims further comprising a spacer located between the binding domain and the trans-splicing domain of the RTM.

16. An RTM according to claim 15 wherein the spacer comprises the sequence of SEQ ID NO: 10 or a variant thereof.

17. An RTM according to any one of the preceding claims that comprises in order in a 5′ to 3′ direction the binding region, the trans-splicing domain and the coding sequence.

18. An RTM according to claim 17 wherein the trans-splicing domain comprises a acceptor site

19. An RTM according to claim 18 wherein the trans-splicing domain comprises the nucleotide sequence of SEQ ID NO: 12 or a variant thereof.

20. An RTM according to any one of claims 17 to 19 further comprising a sequence encoding a 2A self-cleaving peptide, said sequence being located between the trans-splicing domain and the coding sequence of the RTM.

21. An RTM according to claim 20 wherein the 2A cleavage site comprises the sequence of SEQ ID NO: 11 or a variant thereof.

22. An RTM according to any one of claims 17 to 21 wherein the coding sequence for the suicide protein lacks a start codon.

23. An RTM according to any one of claims 17 to 22 comprising the nucleic acid sequence of SEQ ID 2NO: 13; a variant of SEQ ID NO: 13; SEQ ID NO: 15; or a variant of SEQ ID NO: 15.

24. An RTM according to any one of claims 1 to 16 comprising in order in a 5′ to 3′ direction; the coding sequence, trans-splicing domain and the binding region.

25. An RTM according to claim 23 wherein the trans-splicing domain comprises a splice donor site.

26. An RTM according to any one of claim 24 or 25 wherein the coding sequence for the suicide protein comprises a start codon.

27. An RTM according to any one of claims 24 to 26 further comprising a ribozyme sequence located downstream (3′) of the binding domain, wherein said ribozyme sequence cleaves the RTM 3′ of the binding domain to remove downstream nucleotide sequence.

28. A nucleic acid encoding an RTM according to any one of the preceding claims.

29. A nucleic acid encoding an HSV thymidine kinase comprising an amino acid sequence that is a variant of SEQ ID NO: 6 with modifications relative to SEQ ID NO: 6 at positions corresponding to positions 46 and 60 of SEQ ID NO: 6, optionally positions corresponding to positions 1, 46 and 60 of SEQ ID NO: 6.

30. A nucleic acid according to claim 29 wherein the HSV thymidine kinase comprises an amino acid sequence that is variant of SEQ ID NO: 6 with an M to L substitution at a position corresponding to position 46; and an M to I substitution at a position corresponding to position 60 and optionally a deletion of M at a position corresponding to position 1 of SEQ ID NO: 6.

31. A nucleic acid according to claim 29 or 30 wherein the HSV thymidine kinase comprises the amino acid sequence of SEQ ID NO: 8 or a variant thereof.

32. A nucleic acid according to any one of claims 29 to 31 comprising the nucleotide sequence of SEQ ID NO: 9 or a variant thereof.

33. A nucleic acid according to any one of claims 29 to 32 wherein the nucleic acid is an RTM further comprising a binding region specific for a target pre-mRNA and a trans-splicing domain.

34. An expression vector comprising a nucleic acid according to claim 28.

35. An expression vector according to claim 34, wherein the vector is a viral vector.

36. A viral particle comprising a nucleic acid according to claim 28 or an expression vector according to claim 34 or claim 35.

37. An isolated cell comprising an RTM according to any one of claims 1 to 27, a nucleic acid according to claim 28 or an expression vector according to claim 34 or claim 35.

38. A pharmaceutical composition comprising an RTM according to any one of claims 1 to 27, a nucleic acid according to claim 28 or an expression vector according to claim 34 or claim 35, a viral particle according to claim 36 or an isolated host cell according to claim 37.

39. A method of treatment of cancer comprising;
administering an RTM according to any one of claims 1 to 27, a nucleic acid according to claim 28 or an expression vector according to claim 34 or claim 35, a viral particle according to claim 36, an isolated cell according to claim 37 or a pharmaceutical composition according to claim 38, to an individual in need thereof.

40. A composition comprising an RTM according to any one of claims 1 to 27, a nucleic acid according to claim 28 or an expression vector according to claim 34 or claim 35, a viral particle according to claim 36, an isolated host cell according to claim 37 or a pharmaceutical composition according to claim 38, for use in a method of treatment of cancer.

41. Use of an RTM according to any one of claims 1 to 27, a nucleic acid according to claim 28 or an expression vector according to claim 34 or claim 35, a viral particle according to claim 36, an isolated host cell according to claim 37 or a pharmaceutical composition according to claim 38 in the manufacture of a medicament for use in treating cancer.

42. A method according to claim 39 an composition for use according to claim 40 or use according to claim 41, wherein following the administration of the RTM, nucleic acid, expression vector, viral particle, isolated cell or pharmaceutical composition to the individual, the method further comprises, administering to the individual a first treatment with an inactive pro-form of a cytotoxic compound that is activated by the suicide protein, such that the cytotoxic compound is activated in cancer cells in the individual.

43. A method, composition for use, or use according to claim 42, further comprising administering to the individual one or more further treatments with the inactive pro-form, such that the cytotoxic compound is activated in cells that have become or are becoming cancer cells in the individual following said first treatment.

44. A method, composition for use, or use according to claim 43, wherein the one or more further treatments occur at least 1 week after the first treatment.

45. A method of preventing cancer occurrence or recurrence in an individual undergoing cell therapy, the method comprising administering a population of cells according to claim 37 to an individual in need thereof.

46. A method according to claim 45 comprising administering to the individual an inactive pro-form of a cytotoxic compound that is activated by the suicide protein, such that the cytotoxic compound is activated in cells in the population that have become or are becoming cancerous in the individual.

47. A method according to claim 45 or claim 46 wherein the cells are autologous cells from the individual.

48. A method according to any one of claims 45 to 47 wherein the cells are haematopoietic cells.

49. A method according to any one of claims 39 or 42 to 48; composition for use according to any one of claims 40 or 42 to 44 or use according to any one of claims 41 to 44, wherein the cancer is characterised by expression of an HERV gene.

50. A method according to any one of claims 39 or 42 to 49; composition for use according to any one of claim 40, 42 to 44 or 49 or use according to any one of claim 41 to 44 or 49, wherein the cancer is pancreatic or hepatic cancer.

51. A method of killing a cell in vitro comprising;
contacting a cell with an RTM according to any one of claims 1 to 27, a nucleic acid according to claim 28, an expression vector according to claim 34 or claim 35, or a viral particle according to claim 36, such that the cell expresses the suicide protein; and, contacting the cell with the inactive pro-form of a cytotoxic compound that is activated by the suicide protein, such that the cytotoxic compound is activated by the suicide protein, thereby killing the cell.

52. A method according to claim 51, wherein the cell expresses an HERV gene.

53. A method of depleting HERV gene expressing cells in a population comprising;
contacting the population of cells with an RTM according to any one of claims 1 to 27, a nucleic acid according to claim 28, an expression vector according to claim 34 or claim 35, a viral particle according to claim 36 and/or a pharmaceutical composition according to claim 38, such that HERV gene expressing cells in the population of cells express the suicide protein; and,
contacting the population with the inactive pro-form of a cytotoxic compound that is activated by the suicide protein, such that the suicide protein activates the cytotoxic compound in HERV gene expressing cells in the population, thereby depleting HERV gene expressing cells in the population.

54. A method according to claim 53 wherein the HERV gene expressing cells are cancer cells.

55. A method according to claim 53 or 54 wherein the population of cells is a sample of cells obtained from an individual or the descendants thereof.

56. A method according to any one of claims 53 to 55 wherein the cells are haematopoietic cells.


SEQUENCE LISTING (40 sequences)

SEQ ID NO: 1 - DNA, 44 bp, Artificial Sequence
  HERV-K target sequence of binding region
tggataatcctatagaagtatatgttaatgatagcgaatgggta

SEQ ID NO: 2 - RNA, 44 nt, Artificial Sequence
  HERV-K target sequence of binding region
uggauaauccuauagaaguauauguuaaugauagcgaaugggua

SEQ ID NO: 3 - DNA, 44 bp, Artificial Sequence
  Reverse complement of HERV-K target sequence
tacccattcgctatcattaacatatacttctataggattatcca

SEQ ID NO: 4 - RNA, 44 nt, Artificial Sequence
  Reverse complement of HERV-K target sequence
uacccauucgcuaucauuaacauauacuucuauaggauuaucca

SEQ ID NO: 5 - RNA, 44 nt, Artificial Sequence
  Modified reverse complement of HERV-K target sequence
uacucauucgcuaucauuuucauauacuucugugggauuaucca

SEQ ID NO: 6 - protein, 376 aa, Artificial Sequence
  HSV Thymidine Kinase (HSV-tk) amino acid sequence
MASYPCHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 7 - DNA, 1131 bp, Artificial Sequence
  HSV Thymidine Kinase (HSV-tk) coding sequence
atggcttcgtacccctgccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactga

SEQ ID NO: 8 - protein, 375 aa, Artificial Sequence
  Modified HSV Thymidine Kinase (HSV-tk) amino acid sequence
ASYPCHQHASAFDQAARSRGHSNRRTALRPRRQQEATEVRPEQKLPTLLRVYIDGPHGIGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 9 - DNA, 1128 bp, Artificial Sequence
  Modified HSV Thymidine Kinase (HSV-tk) coding sequence
gcttcgtacccctgccatcaacacgcgtctgcgttcgaccaggctgcgcgttctcgcggccatagcaaccgacgtacggcgttgcgccctcgccggcagcaagaagccacggaagtccgcccggagcagaaactgcccacgctactgcgggtttatatagacggtccccacgggatcgggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactga

SEQ ID NO: 10 - DNA, 39 bp, Artificial Sequence
  Spacer sequence
ggtgacgaaaacgtgctatcagttcgctcccccactccc

SEQ ID NO: 11 - protein, 22 aa, Artificial Sequence
  P2A cleavage sequence
GSGATNFSLLKQAGDVEENPGP

SEQ ID NO: 12 - DNA, 51 bp, Artificial Sequence
  Trans-splicing domain sequence
gggcctgggcctgggtactaacacgatcgtttttttccctttttttccagg

SEQ ID NO: 13 - RNA, 1511 nt, Artificial Sequence
  RTM sequence
gcuagcuacucauucgcuaucauuuucauauacuucugugggauuauccaccucgcggugacgaaaacgugcuaucaguucgcucccccacucccgcuuucauuuuugucuugucuuuuuuuaaccugggccugggccuggguacuaacacgaucguuuuuuucccuuuuuuuccaggggaagcggagcuacuaacuucagccugcugaagcaggcuggagacguggaggagaacccugggccugcuucguaccccugccaucaacacgcgucugcguucgaccaggcggcgcgaucacggggacacagcaaccgacggacggcguugcgcccucgccggcagcaagaagccacggaaguccgcccggagcagaaaaugcccacgcuacugcggguuuauauagacgguccccacgggauggggaaaaccaccaccacgcaacugcugguggcccuggguucgcgcgacgauaucgucuacguacccgagccgaugacuuacuggcgggugcugggggcuuccgagacaaucgcgaacaucuacaccacacaacaccgccucgaccagguaaguaucaagguuacaagacagguuuaaggagaccaauagaaacugggcuugucgagacagagacgacucuugcguuucugauaggcaccuauuggucuuacugacauccacuuugccuuucucuccacagggugagauaucggccggggacgcggcggugguaaugacaagcgcccagauaacaaugggcaugccuuaugccgugaccgacgccguucuggcuccucauaucgggggggaggcugggagcucacaugccccuccuccggcccucacccucaucuucgaccgccaucccaucgccgcccuccugugcuacccggccgcgcgauaccuuaugggcagcaugaccccccaggccgugcuggcguucguggcccucaucccgccgaccuugcccggcacaaacaucguguugggggcccuuccggaggacagacacaucgaccgccuggccaaacgccagcgccccggcgagcggcuugaccuggcuaugcuggccgcgauucgccgcguuuacgggcugcuugccaauacggugcgguaucugcagggcggcgggucguggcgggaggauuggggacagcuuucggggacggccgugccgccucagggugccgagccucagagcaacgcgggcccacgaccccauaucggggacacguuauuuacccuguuucgggcccccgaguugcuggcccccaacggcgaccuguauaacguguuugccugggccuuggacgucuuggccaaacgccuccgucccaugcacgucuuuauccuggauuacgaccaaucgcccgccggcugccgggacgcccugcugcaacuuaccuccgggaugguccagacccacgucaccaccccaggcuccauaccgacgaucugcgaccuggcgcgcacguuugcgcgggagaugggggaggcgaacugacucgag

SEQ ID NO: 14 - DNA, 1511 bp, Artificial Sequence
  Coding DNA sequence for RTM
gctagctactcattcgctatcattttcatatacttctgtgggattatccacctcgcggtgacgaaaacgtgctatcagttcgctcccccactcccgctttcatttttgtcttgtctttttttaacctgggcctgggcctgggtactaacacgatcgtttttttccctttttttccaggggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgcccctcctccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgcctcagggtgccgagcctcagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgactcgag

SEQ ID NO: 15 - RNA, 1511 nt, Artificial Sequence
  RTM sequence with modified HSV-tk coding sequence
gcuagcuacucauucgcuaucauuuucauauacuucugugggauuauccaccucgcggugacgaaaacgugcuaucaguucgcucccccacucccgcuuucauuuuugucuugucuuuuuuuaaccugggccugggccuggguacuaacacgaucguuuuuuucccuuuuuuuccaggggaagcggagcuacuaacuucagccugcugaagcaggcuggagacguggaggagaacccugggccugcuucguaccccugccaucaacacgcgucugcguucgaccaggcggcgcgaucacggggacacagcaaccgacggacggcguugcgcccucgccggcagcaagaagccacggaaguccgcccggagcagaaacugcccacgcuacugcggguuuauauagacgguccccacgggaucgggaaaaccaccaccacgcaacugcugguggcccuggguucgcgcgacgauaucgucuacguacccgagccgaugacuuacuggcgggugcugggggcuuccgagacaaucgcgaacaucuacaccacacaacaccgccucgaccagguaaguaucaagguuacaagacagguuuaaggagaccaauagaaacugggcuugucgagacagagacgacucuugcguuucugauaggcaccuauuggucuuacugacauccacuuugccuuucucuccacagggugagauaucggccggggacgcggcggugguaaugacaagcgcccagauaacaaugggcaugccuuaugccgugaccgacgccguucuggcuccucauaucgggggggaggcugggagcucacaugccccuccuccggcccucacccucaucuucgaccgccaucccaucgccgcccuccugugcuacccggccgcgcgauaccuuaugggcagcaugaccccccaggccgugcuggcguucguggcccucaucccgccgaccuugcccggcacaaacaucguguugggggcccuuccggaggacagacacaucgaccgccuggccaaacgccagcgccccggcgagcggcuugaccuggcuaugcuggccgcgauucgccgcguuuacgggcugcuugccaauacggugcgguaucugcagggcggcgggucguggcgggaggauuggggacagcuuucggggacggccgugccgccucagggugccgagccucagagcaacgcgggcccacgaccccauaucggggacacguuauuuacccuguuucgggcccccgaguugcuggcccccaacggcgaccuguauaacguguuugccugggccuuggacgucuuggccaaacgccuccgucccaugcacgucuuuauccuggauuacgaccaaucgcccgccggcugccgggacgcccugcugcaacuuaccuccgggaugguccagacccacgucaccaccccaggcuccauaccgacgaucugcgaccuggcgcgcacguuugcgcgggagaugggggaggcgaacugacucgag

SEQ ID NO: 16 - DNA, 1511 bp, Artificial Sequence
  Coding DNA sequence for RTM with modified HSV-tk coding sequence
gctagctactcattcgctatcattttcatatacttctgtgggattatccacctcgcggtgacgaaaacgtgctatcagttcgctcccccactcccgctttcatttttgtcttgtctttttttaacctgggcctgggcctgggtactaacacgatcgtttttttccctttttttccaggggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaactgcccacgctactgcgggtttatatagacggtccccacgggatcgggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgcccctcctccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgcctcagggtgccgagcctcagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgactcgag

SEQ ID NO: 17 - DNA, 66 bp, Artificial Sequence
  Nucleotide sequence encoding P2A peptide
ggaagcggagctactaacttcagcctgctgaagcaggctggagacgtggaggagaaccctgggcct

SEQ ID NO: 18 - DNA, 11 bp, Artificial Sequence
  BbvCI restriction enzyme site
cctcagcagtg

SEQ ID NO: 19 - DNA, 10 bp, Artificial Sequence
  BbvCI restriction enzyme site
cctcgcggtg

SEQ ID NO: 20 - RNA, 19 nt, Artificial Sequence
  Polypyrimidine tract
uuuuuuucccuuuuuuucc

SEQ ID NO: 21 - DNA, 15 bp, Artificial Sequence
  Intronic splice enhancer
gggcctgggcctggg

SEQ ID NO: 22 - DNA, 11 bp, Artificial Sequence
  Splice donor site
cagguaagtat

SEQ ID NO: 23 - DNA, 52 bp, Artificial Sequence
  Site-directed mutagenesis forward primer
gaaactgcccacgctactgcgggtttatatagacggtccccacgggatcggg

SEQ ID NO: 24 - DNA, 52 bp, Artificial Sequence
  Site-directed mutagenesis reverse primer
cccgatcccgtggggaccgtctatataaacccgcagtagcgtgggcagtttc

SEQ ID NO: 25 - DNA, 31 bp, Artificial Sequence
  Mutagenesis forward primer tktr 3 mut
acggtccccacgggatcgggaaaaccaccac

SEQ ID NO: 26 - DNA, 31 bp, Artificial Sequence
  Mutagenesis reverse primer tktr 3 mut
gtggtggttttcccgatcccgtggggaccgt

SEQ ID NO: 27 - DNA, 37 bp, Artificial Sequence
  XbaI forward  primer
gacattgattattgtctagaactagttgagccccacg

SEQ ID NO: 28 - DNA, 37 bp, Artificial Sequence
  XbaI reverse primer
cgtggggctcaactagttctagacaataatcaatgtc

SEQ ID NO: 29 - DNA, 34 bp, Artificial Sequence
  XhoI forward primer
gggaggcgaactgactcgagaacttgtttattgc

SEQ ID NO: 30 - DNA, 34 bp, Artificial Sequence
  XhoI reverse primer
gcaataaacaagttctcgagtcagttcgcctccc

SEQ ID NO: 31 - DNA, 28 bp, Artificial Sequence
  TG BcuI forward primer
gcactagttgcatgtataatttctacag

SEQ ID NO: 32 - DNA, 28 bp, Artificial Sequence
  TG NheI reverse primer
cggctagcttatagcatgtcctgtattg

SEQ ID NO: 33 - DNA, 20 bp, Artificial Sequence
  Psi forward primer
caggactcggcttgctgaag

SEQ ID NO: 34 - DNA, 20 bp, Artificial Sequence
  Psi reverse primer
tcccccgcttaatactgacg

SEQ ID NO: 35 - DNA, 22 bp, Artificial Sequence
  Albumin forward primer
gctgtcatctcttgtgggctgt

SEQ ID NO: 36 - DNA, 21 bp, Artificial Sequence
  Albumin reverse primer
actcatgggagctgctggttc

SEQ ID NO: 37 - DNA, 20 bp, Artificial Sequence
  WPRE forward primer
ggcactgacaattccgtggt

SEQ ID NO: 38 - DNA, 20 bp, Artificial Sequence
  WPRE reverse primer
agggacgtagcagaaggacg

SEQ ID NO: 39 - DNA, 23 bp, Artificial Sequence
  WPRE probe
acgtcctttccatggctgctcgc

SEQ ID NO: 40 - DNA, 44 bp, Artificial Sequence
  Binding domain sequence of targeting Np9
tactcattcgctatcattttcatatacttctgtgggattatcca